SEQUENCE LISTING (8 sequences)

SEQ ID NO: 1 - DNA, 160 bp, Artificial Sequence
  Illumina P7 Adaptor
  misc_feature (75)..(84): n is a, c, g, or t
acgatgagcagagccagaaccagaaggaacttgactctagagatcggaagagcacacgtctgaactccagtcacnnnnnnnnnngtcctcatctgagagctactcatcaacggtatctcgtatgccgtcttatgcttgttaattaagaattcctggacga

SEQ ID NO: 2 - DNA, 62 bp, Artificial Sequence
  primer
  misc_feature (30)..(35): n is a, c, g, or t
aatgatacggcgaccaccgagatctacacnnnnnncgagggcctatttcccatgattccttc

SEQ ID NO: 3 - DNA, 39 bp, Artificial Sequence
  primer
caagcagaagacggcatacgagataccgttgatgagtag

SEQ ID NO: 4 - DNA, 62 bp, Artificial Sequence
  primer
aatgatacggcgaccaccgagatctacacgagtggcgagggcctatttcccatgattccttc

SEQ ID NO: 5 - DNA, 39 bp, Artificial Sequence; identical to SEQ ID NO: 3
  primer

SEQ ID NO: 6 - DNA, 22 bp, Artificial Sequence
  Pac-I Fragment
gcctttaagccaatgctagctg

SEQ ID NO: 7 - DNA, 25 bp, Artificial Sequence
  Pac-I Fragment
gtaaatggacagagggtgtttaacc

SEQ ID NO: 8 - DNA, 44 bp, Artificial Sequence
  primer
cgatttcttggctttatatatcttgtggaaaggacgaaacaccg

(19) United States
(12) Patent Application Publication
(10) Pub. No.: US 2021/0189352 A1
(43) Pub. Date: Jun. 24, 2021

(54) ENHANCED REPROGRAMMING OF SOMATIC CELLS

(71) Applicant: IMBA - INSTITUT FÜR MOLEKULARE BIOTECHNOLOGIE GMBH, Wien (AT)

(72) Inventors: Ulrich ELLING, Perchtoldsdorf (AT); Elena BUDUSAN, Dutton Park (AU); Georg MICHLITS, Vienna (AT); Cecilia RAUPACH, Chemnitz (DE); Gintautas VAINORIUS, Vienna (AT); Szu-Hsein WU, Szu-Hsien (AT)

(21) Appl. No.: 16/755,862

(22) PCT Filed: Oct. 12, 2018
(86) PCT No.: PCT/EP2018/077936

§ 371 Date: Apr. 13, 2020

(30) Foreign Application Priority Data
Oct. 13, 2017 (EP) 17196419.0

(51) Int. Cl. C12N 5/074; C12N 15/113

(52) U.S. Cl. CPC C12N 5/0696; C12N 15/113; C12N 2501/603; C12N 2501/602; C12N 2501/605; C12N 2501/999; C12N 2501/606; C12N 2501/608; C12N 2310/141; C12N 2310/11; C12N 2501/604

(57) ABSTRACT

A method of preparing a population of iPS cells including (i) expressing one or more Yamanaka factors selected from Oct3/4, Sox2, Klf4, Myc, Nanog and Lin28, and reducing the amount and/or activity of Menin (Men1) in a population of target cells, and (ii) optionally isolating the iPS cells from the target cell population; and a method of enhanced differentiation of a first cell into a somatic cell of a tissue of interest, including (i) treating a cell with a differentiation factor of the tissue of interest, and (ii) reducing the amount and/or activity of Menin (Men1) in a population of target cells.

The present invention relates to methods for improving the efficiency of induced pluripotent stem cell (iPS) formation.

### BACKGROUND OF THE INVENTION

Since its discovery, cellular reprogramming to pluripotency has become a broadly used experimental tool. Beyond its great utility in basic and biomedical research, induced pluripotent stem cell (iPSC) reprogramming is believed to be applicable for a wide range of medical applications such as the generation of patient-specific tissue for cellular therapy. However, the process of iPSC reprogramming remains very inefficient and stochastic in nature, which diminishes its utility for many applications, particularly if the source of somatic cells is limited. While the major roadblock preventing efficient iPS reprogramming is thought to lie in the hard-wired epigenetic landscape, the key mechanisms and factors contributing to this roadblock remain incompletely understood.

Guo et al., Stem Cell Research 18, 2017, pp. 67-69, describes discloses iPS cells expressing Oct4 and Nanog that had a point mutation in exon9 of Men1.

Parekh et al., International Journal of Endocrinology, 2015, pp. 1-10, discloses the differentiation of 3T3-L1 cells into adipocytes.

Aziz et al., Developmental Biology 332, 2009, pp. 116-130, discloses the differentiation of C2C12 myoblasts and C3H10T1 fibroblasts into myotubes.

Improvements in iPS reprogramming were described in WO 2016/012544 A2, in particular, the preparation of a population of iPS cells by reducing the amount of components of the SUMO pathway. Yet there still is a need for more versatile or efficient reprogramming enhancers.

There exists a need in the art for improved methods for reprogramming mammalian cells. It is therefore a goal to provide improved methods for the generation of iPS cells.

### SUMMARY OF THE INVENTION

The invention improves reprogramming of somatic cells into iPS cells by reducing the amount and/or activity of Menin (Men1) in addition to the expression of Yamanaka factors.

Accordingly, provided is a method of preparing a population of iPS cells comprising (i) expressing one or more Yamanaka factors selected from Oct3/4, Sox2, Klf4, Myc, Nanog and Lin28, and reducing the amount and/or activity of Menin (Men1) in a population of target cells, and (ii) optionally isolating the iPS cells from the target cell population. Reduction of expression or activity of Menin may be by using one or more agents that either inhibits the expression of Menin or that inhibit the activity of Menin. In certain embodiments, the expression of the one or more Yamanaka factors as well as the inhibition of expression or activity of Menin is preferably transient, i.e. reversible.

In addition to Menin, further, alternative or combinable, reprogramming factors were found, i.e. Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and Trp53. These reprogramming factors, including Men1, are also referred to as “roadblocks of reprogramming”. Their inhibition can facilitate or enhance cell reprogramming in the generation of iPS cells. Accordingly, the invention also provides a method of preparing a population of iPS cells comprising reducing the amount and/or activity of Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53 or a combination thereof in a population of target cells. The Yamanaka may be expressed as described for Menin. The iPS cells may be isolated from the target cell population as described for Menin. In general, anything described herein for Menin, which is the preferred reprogramming target, also applies to these other reprogramming targets (Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53).

The invention also relates to a method for preparing a population of differentiated cells, comprising (i) preparing a population of iPS cells by expressing one or more Yamanaka factors selected from Oct3/4, Sox2, Klf4, Myc, Nanog and Lin28, and reducing the amount and/or activity of Menin (Men1) and/or one or more of said other reprogramming targets in a population of target cells, and (ii) differentiating the iPS cells using a protocol or factor to form a population of differentiated cells.

In another aspect, the present invention provides a population of iPS cells that is prepared according to the inventive method of enhanced iPS cell reprogramming. In contrast to currently available iPS cells, the iPS cells of the present invention have reduced or no Menin function and/or reduced or no function of one or more of the other reprogramming targets.

In yet another aspect, the invention also includes a cell culture media for the enhanced iPS cells reprogramming protocol comprising Yamanaka factors or Yamanaka-inducing agents and one or more agents that inhibit the expression or activity of Menin and/or one or more agents that inhibit one or more of the other reprogramming targets.

Also provided is a kit suitable for performing the method of the invention comprising target cells and the cell medium according to the present invention. Optionally the kit further includes one or more agents for differentiating the iPS cells of the present invention. A kit may comprise one or more Yamanaka factors or Yamanaka-inducing agents and one or more agents that inhibit the expression, translation or activity of Menin and/or one or more agents that inhibit one or more of the other reprogramming targets, preferably wherein said Yamanaka factors or Yamanaka-inducing agents and one or more agents that inhibit the expression, translation or activity of Menin and/or one or more agents that inhibit one or more of the other reprogramming targets are in one or more cell culture medium.

All embodiments of the invention are described together in the following detailed description and all preferred embodiments relate to all embodiments, aspects, methods and kits alike. E.g. preferred and detailed descriptions of the inventive methods read alike on suitability's and requirements of the inventive kits. Descriptions of the kits or its parts read on components that can be used in the inventive methods. All embodiments can be combined with each other, except where otherwise stated.

### DETAILED DESCRIPTION OF THE INVENTION

From a functional genetic screen to systematically identify factors involved in preventing iPS reprogramming, the inventors uncovered new roadblocks for reprogramming somatic cells to pluripotent stem cells. Reprogramming is a time-consuming process and suffers from low efficiency, therefore limiting the clinical applications of iPSCs. The standard protocol for reprogramming somatic cells into iPSCs is the ectopic expression of a set of core pluripotency-related transcription factors, the so-called Yamanaka factors including Oct4, Sox2, Klf and c-Myc, modifiers such as Lin28 and Nanog, p53 knockdown, and the substitution of L-Myc for c-Myc. Reprogramming efficiency into a pluripotent state varies widely, depending on the cell type to be reprogrammed, the factors used to reprogram and on the dosage of said factors. Generally, reprogramming efficiency with said Yamanaka factors is below 1% of treated cells. Surprisingly it was found that inhibition of the newly identified genetic roadblock factors led to a dramatic increase in cell reprogramming efficacy. The present invention therefore identified these roadblock factors as a target to enhance iPSC reprogramming. One such roadblock factor identified by the inventors of the present application is Menin (Men1). Further reprogramming targets, that can be targeted like Menin, in addition or as an alternative to Menin are Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53 (also referred to as “other reprogramming targets” herein). Menin is the most preferred reprogramming target of the invention and therefore much is described with regard to Menin herein. Nevertheless, anything described herein with regard to Menin also applies to these other reprogramming targets. So far, it had not previously been known that negative regulation of Menin or the other reprogramming targets in combination with the expression of a subset of Yamanaka factors in a population of target cells would lead to such a dramatic increase in cell reprogramming efficiency.

The present invention therefore provides a method of preparing a population of iPS cells comprising (i) expressing one or more Yamanaka factors selected from Oct3/4, Sox2, Klf4, Myc, Nanog and Lin28, and (ii) reducing the amount and/or activity of Menin (Men1) and/or of one or more of said other reprogramming targets, in a population of target cells. Similarly, the present invention relates to a method of preparing an iPS cell comprising (i) expressing one or more Yamanaka factors selected from Oct3/4, Sox2, Klf4, Myc, Nanog and Lin28, and (ii) reducing the amount and/or activity of Menin (Men1), and/or one or more of said other reprogramming targets, in a population of target cells. In some embodiments of the present invention, the method further comprises isolating the iPS cell or iPS cell population from the target cell population.

Human Menin (Men1 gene) is listed in the NCBI database as GeneID: 4221. The entry for Men1 includes information including amino acid and nucleic acid sequences (www.ncbi.nlm.nih.gov/gene?Db=gene&Cmd=DetailsSearch&Term=4221) as of the filing date of the present application. It encodes a protein of 615 amino acid residues and functions as a transcriptional regulator by interacting with a variety of proteins including transcription factors JUND, NFKB and SMADs. Menin is a putative tumour suppressor associated with a syndrome known as multiple endocrine neoplasia type 1. It is also an essential component of a MLL/SET1 histone methyltransferase (HMT) complex, a complex that specifically methylates Lys-4 of histone H3 (H3K4).

Sequences and full descriptions of the other reprogramming targets can be found in publicly available databases, such as GeneCards (www.genecards.org). The GeneCards database comprises a compilation of information available in other databases such as the NCBI database or the EBI database. Of course, these other databases may be consulted as well. For example, Socs3 is also known as “Suppressor Of Cytokine Signaling 3”, Eif4e2 as “Eukaryotic Translation Initiation Factor 4E Family Member 2”, Apc as “APC, WNT Signaling Pathway Regulator”, Setd2 as “SET Domain Containing 2”, Axin1 as “Axin 1”, Cdk13 as “Cyclin Dependent Kinase 13”, Psip1 as “PC4 And SFRS1 Interacting Protein 1”, Cabin as “Calcineurin Binding Protein 1”, Fbxw7 as “F-Box And WD Repeat Domain Containing 7”, Tcf711 as “Transcription Factor 7 Like 1”, Tlk2 as “Tousled Like Kinase 2”, Hira as “Histone Cell Cycle Regulator”, Uba2 as “Ubiquitin Like Modifier Activating Enzyme 2”, Ubr4 as “Ubiquitin Protein Ligase E3 Component N-Recognin 4”, Sae1 as “SUMO1 Activating Enzyme Subunit 1”, Chaf1a as “Chromatin Assembly Factor 1 Subunit A”, Asf1a as “Anti-Silencing Function 1A Histone Chaperone”, Dot1l as “DOT1 Like Histone Lysine Methyltransferase”, Pias1 as “Protein Inhibitor Of Activated STAT 1”, Pten as “Phosphatase And Tensin Homolog”, Senp1 as “SUMO1/Sentrin Specific Peptidase 1”, Trp53 as “Tumor Protein P53”.

It is the aim of the inventive method to inhibit endogenous Menin function or function of one or more of the other reprogramming targets in somatic cells. Functional inhibition may be achieved by reducing the amount and/or activity of Menin and/or of one or more of the other reprogramming targets. One way of reducing the amount of a protein in the cell is the inhibition of endogenous gene expression or translation. Accordingly, in one embodiment of the present invention the reduction of the amount of Menin and/or of one or more of the other reprogramming targets comprises administering to the target cells one or more agents that inhibit the expression or translation of Menin and/or of one or more of the other reprogramming targets. Preferably, the one or more agents inhibiting the expression of Menin and/or of one or more of the other reprogramming targets are inhibitory nucleic acids.

Inhibitory nucleic acids act through inhibiting gene expression or the translation of nucleic acids, e.g. by a process called gene silencing or RNA interference (RNAi). Inhibitory nucleic acids usually comprise a complementary nucleic acid sequence to Men1 or the Men1 transcript for its inhibition that comprises a step of hybridization thereto. Examples of such inhibitory nucleic acids are siRNAs, shRNAs, sgRNA, miRNAs and antisense nucleic acids, i.e. antisense RNA, DNA or a chemical analogue, like LNA (locked nucleic acid) or PNA (peptide nucleic acid). Accordingly, the inhibitory nucleic acid according to a preferred embodiment of the present invention is an inhibitory siRNA, shRNA, sgRNA, miRNA or antisense nucleic acid. If Menin inhibition in the target cell is aimed to be non-transient, but stable, sgRNAs combined with CRISPR-Cas9 may be used. However, also transient inhibition is possible via CRIPSR-Cas, e.g. by using a modified Cas enzyme, such as dCas. Such a Cas enzyme may be unable to cut the target gene but inhibits its expression by binding to the target gene. Anti-Menin siRNAs, shRNAs, sgRNA, miRNAs, antisense nucleic acids or sgRNAs according to the present invention or such inhibitory nucleic acids against the other reprogramming factors are either commercially available or may be designed according to standard RNAi design tool known to the skilled person (e.g. siRNA Wizard (InvivoGen) or BLOCK-iT RNAi Designer (ThermoFisher Scientific)).

Methods for silencing genes by transfecting cells with inhibitory nucleic acids or constructs encoding said nucleic acids are known in the art. To express an RNAi agent in somatic cells, a nucleic acid construct comprising a sequence that encodes the RNAi agent, operably linked to suitable expression control elements, e.g., a promoter, may be introduced into the cells as known in the art. For purposes of the present invention a nucleic acid construct that comprises a sequence that encodes a nucleic acid or polypeptide of interest, the sequence being operably linked to expression control elements such as a promoter that direct transcription in a cell of interest, is referred to as an “expression cassette”. The promoter can be an RNA polymerase I, II, or III promoter functional in somatic mammalian cells. In certain embodiments expression of the RNAi agent is conditional, e.g. by requiring an extrinsic signal or factor to initiate expression. In some embodiments expression is regulated by placing the sequence that encodes the RNAi agent under control of a conditional, hence regulatable (e.g. inducible or repressible) promoter. One of skill in the art will be able to identify inhibitory nucleic acid sequences that target corresponding regions of human orthologs.

Suppression of Menin expression or translation according to the present invention or of expression or translation of any other reprogramming target may only be transient, i.e. temporary. Various transient gene silencing systems are commercially available and known to the skilled person in the art. Transient suppression can for example be achieved through transient delivery methods or stable delivery of conditional expression cassettes.

As can be appreciated by the skilled person, examples of transient delivery methods include transient transfection of inhibitory nucleic acid molecules, transient transfection of DNA or RNA vectors encoding inhibitory nucleic acid expression cassettes, infection with non-integrating viruses (e.g. AAV, Adenovirus, Sendaivirus and many others) encoding inhibitory nucleic acids or other inhibitory genetic elements to suppress the target. A further option is an episomal vector, such as a plasmid.

There are also many examples of how to stably deliver inducible/conditional expression cassettes into mammalian cells, e.g. retro-/lentiviruses, the CRISPR and TALEN technologies, and other delivery methods. sgRNAs are preferably used in a CRISPR-Cas setting. The sgRNA may be complexed by a Cas enzyme and used to edit and preferably inactivate a Menin gene.

Therefore, in a preferred embodiment of the invention, the agent inhibiting Menin (or the other reprogramming factors) expression or translation is an inhibitory siRNA, shRNA, sgRNA, miRNA or antisense nucleic acid encoded by a transient expression system in the target cells.

In an embodiment of the invention, a self-inactivating retroviral vector encoding a shRNA under control of a Tet-responsive element promoter (TRE3G) may be used. That vector encoded a shRNA to be used in the method of the invention to suppress the expression of one or more reprogramming roadblock factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53, preferably Menin, in a population of target cells. The vector preferably provides for inducible and reversible expression of the shRNA. A possible vector is pSIN-TRE3G-mCherry-miRE-PGK-Neo. However, the skilled person would readily be able to identify other suitable transient inducible/regulatable expression cassettes which can be adapted to encode a shRNA molecule to be used in the method of the invention, and also the protocol used to introduce that vector to a population of target cells.

In some embodiments of the present invention, cells are contacted with one or more agents inhibiting the expression or translation of Menin or of the other reprogramming factors for a time period of at least 1 days, while in other embodiments the period of time is at least 3, 5, 10, 15, or 20 days or more or any range in between these day numbers. In some embodiments, cells are contacted for at least 1 and no more than 3, 5, 10, 15, or 20 days.

A further embodiment of the invention is wherein the target cell is exposed to a transient expression system expressing the RNAi agent for 120, 96, 72, 48 or 36 hours or any time in between these hours. As can be appreciated, the above listed range of hours is not intended to be exhaustive, and merely for expediency all time points between the ranges of the hours provided are included in the scope of the method of the invention. For example, in some embodiments the target cell may be exposed to a transient expression system expressing the inhibitory nucleic acid for between 36 to 120 hours.

Apart from reducing the amount of Menin (or of the other reprogramming factors) expression or translation, inhibition of endogenous Menin (or the other endogenous reprogramming factors) function according to the present invention may also be achieved by reducing the activity of the protein. Accordingly, in some embodiments of the inventive method for enhanced iPS reprogramming, the step of reducing the activity of Menin (or of the other reprogramming factors) comprises administering to the cells one or more agents that inhibit the activity of Menin (or of the other reprogramming factors). Preferably, agents inhibiting the activity of Menin according to the present invention (or of the other reprogramming factors) are selected from antibodies, Menin ligands (or ligands to the other reprogramming factors), inhibitory mimics of Menin or of the other reprogramming factors, aptamers or small molecule inhibitors, especially small molecule inhibitors. In some embodiments, the agent (e.g. antibody, ligand, mimic, aptamer or small molecule) binds to and inhibits its target, e.g. Menin (or the other reprogramming factors), or binds to and inhibits a protein whose activity is needed for the target. Small molecule inhibitors of the target complex or pathway components may be used in various embodiments of the invention. Menin ligands and mimics are for example disclosed in Borkin et al., Cancer Cell, 2015, 27: 1-14 and in Grembecka et al., Nat Chem Biol 8(3), 2012: 277-284 and include peptides binding at a Menin binding site as visualized by Borkin in a 3D protein model. Menin ligands are compounds that bind to Menin and inhibit the function of menin. Likewise, menin mimics bind to a Menin-binding site of a Menin-binding partner, without Menin activity, i.e. they block a Menin binding site, e.g. competitively, and prevent access of Menin to its binding partners, like Mll1 (Liu et al., Cell Division (2016) 2, 16036). The ligands and mimics may be proteins, peptides or peptidomimetics such as MI-136, MI-463 or MI-503 as disclosed by Borkin et al. A protein or peptide inhibitor may be a Menin-analogue, which comprise the binding site of Menin but lack further Menin activity (i.e. inhibition of dedifferentiation). Such ligands and mimics can be selected due to binding to the binding pockets of Menin and the Menin binding partners. The inhibitor may disrupt the menin-MLL-AF9 interaction as disclosed in Grembecka et al. (supra). A menin mimic may be an inactivated menin, such as menin comprising mutations M278K and/or Y232K corresponding to human menin as disclosed by Grembecka et al. (supra) and Murai et al. (2011). J Biol Chem. 286(36):31742-8. Murai et al., in particular the sequence alignment of FIG. 1 therein, is incorporated herein by reference, and shows the positions of corresponding amino acids in human Menin. Preferably, a menin mimic of the invention binds MLL or other menin binding partners but lacks M278 and/or Y232 of wild-type menin. Menin inhibitors may also be selected from menin finding fragments of MLL, such as MBM1 and MBM2 or comprising residues 4-15 of MLL, while lacking at least 50%, for example, of the residues of wild-type MLL (Murai et al., supra). MLL or menin and its fragments or mimics should by of the same organism as the cell that is treated. Activity, in particular, prevented activity due to inhibition of Menin, can be easily tested in a test system of reduced inhibition of transdifferentiation or de-differentiation to iPS cells as shown in the examples, especially examples 15-17. Such a test comprises treating a cell with Yamanaka factors or a differentiation factor. This de- or trans-differentiation will be compared with the same set up but with inhibition of Menin using the candidate Menin inhibitor. Increased rate of de- or transdifferentiation indicates a Menin inhibitor. Small molecule inhibitors and peptides may be introduced into target cells by contacting the cells. Peptides and proteins, such as menin mimics are preferably introduced by expression vectors as further detailed below. In preferred embodiments, peptide inhibitors have a size of 3-200 amino acids in length, preferably of 4 to 150 amino acids, or of 5 to 100 amino acids, 6 to 50 amino acids or 7 to 30 amino acids in length.

Inhibition of Menin (or of the other reprogramming factors) activity may be direct or indirect. In another embodiment of the present invention inhibition of Menin (or of the other reprogramming factors) activity may comprise inhibiting Menin protein-protein-interactions (PPIs) (or interactions of the other reprogramming factors), e.g. by inhibiting binding of Menin to FANCD2, GFAP, RPA2, VIM, TCF7L2, RBBP5, ASH2L, HIST3H3, CTNNB1, MLL2, IQGAP1, LEF1, SMAD3, GAST, JUND or NFKB1 (see gene-cards.org or other databases for full names). Preventing such PPIs may result in functional inhibition and/or protein degradation. The Menin inhibitor may also decrease Menin interference with the MLL/SET1 histone methyltransferase (HMT) complex. Therefore, suitable Menin inhibitors that may be used according to the inventive methods (or inhibitors of the other reprogramming factors) include small molecule inhibitors that compete with the binding of an intracellular protein partner or allosteric inhibitors capable of inducing a conformational change leading to a loss of interaction with the binding partners. In some embodiments of the present invention, the Menin inhibitor (or inhibitors of the other reprogramming factors) may be tested and validated prior its use in the inventive method. Experimental techniques to analyze PPIs and protein-inhibitor interactions as known in the art can be used. Generally, an assay includes reprogramming a somatic cell to an iPS cells according to the invention, e.g. by providing a Yamanaka factor as control and as test run the Yamanaka factor and the Menin inhibitor or (or inhibitors of the other reprogramming factors). An increase rate of reprogramming indicates an effective inhibitor.

Examples of inhibitors that may be used for inhibiting the activity of Menin according to the present invention include, but are not limited to, small molecule inhibitors, in particular the inhibitors MI-1 (Grembecka et al., Nat Chem Biol 8(3), 2012: 277-284), KO-382, MI-3, MI-2 (Mol Cell Biol, 2015, 36(4):615-27), MI-2-2, MI-136, MI-372, MI-389, MI-405, MI-463, MI-503 (Borkin et al., Cancer Cell, 2015, 27: 1-14), Vinpocetine, MI-136 and Sinomenine (Int Immunopharmacol, 2016, 40:492).

Further inhibitors to Menin or to the other reprogramming factors (Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53) are available in the art. A database collecting inhibitor information is found at www.selleckchem.com. Inhibitors for Eif4e2 are e.g. 4E1RCat, 4EGI-1 or SBI-0640756; an inhibitor for Apc is Tosyl-L-Arginine Methyl Ester (TAME); inhibitors for Dot1l are Pinometostat (EPZ5676), SGC 0946, EPZ004777; inhibitors for Pten are VO-Ohpic, SF1670; many inhibitors are known for TRP53, such as Pifithrin, in particular Pifithrin-α or Pifithrin-β.

In some embodiments the concentration of the agent inhibiting Menin activity (or other reprogramming factors) added to the medium is between 10 and 10,000 ng/ml, e.g., between 100 and 5,000 ng/ml, e.g., between 1,000 and 2,500 ng/ml or between 2,500 and 5,000 ng/ml, or between 5,000 and 10,000 ng/ml.

Methods of the invention may include treating the cells with multiple agents either concurrently (i.e., during time periods that overlap at least in part) or sequentially and/or repeating the steps of treating the cells with an agent. The agent used in the repeating treatment may be the same as, or different from, the one used during the first treatment.

The cells may be contacted with a reprogramming agent for varying periods of time. In some embodiments the cells are contacted with the agent for a period of time between 1 hour and 60 days, e.g., between 10 and 30 days, e.g., for about 15-20 days. Reprogramming agents may be added each time the cell culture medium is replaced. The reprogramming agent(s) may be removed prior to performing a selection to enrich for pluripotent cells or assessing the cells for pluripotency characteristics.

Other roadblock factors identified by the inventors of the present application resulting in enhanced iPS reprogramming when inhibited are Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53. Accordingly, the present invention also provides a method of preparing a population of iPS cells comprising (i) expressing one or more Yamanaka factors selected from Oct3/4, Sox2, Klf4, Myc, Nanog and Lin28, and reducing the amount and/or activity of Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten and/or Senp1, Trp53 in a population of target cells, and (ii) optionally isolating the iPS cells from the target cell population.

The method according to the present invention may further be improved by reducing the amount and/or activity of more than one reprogramming roadblock factor as identified by the present inventors. Therefore, the present invention also provides a method of preparing a population of iPS cells comprising (i) expressing one or more Yamanaka factors selected from Oct3/4, Sox2, Klf4, Myc, Nanog and Lin28, and reducing the amount and/or activity of two or more factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53.

The above- and below-described embodiments of the inventive method relating to the step of reducing the amount and/or activity of Menin similarly apply to the other reprogramming roadblock factors selected from Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1, Trp53.

The reduction of the amount or activity of Menin (Men1) or the other reprogramming or roadblock factors is preferably an ablation of the amount or activity of the factor. In further embodiments, the reduction does not require absolute abolishing Menin amounts or activity or the amounts of the respective other factors. A reduction of Menin (or respective other factor) net amount or activity, such as a reduction by at least 20%, by at least 30%, by at least 40%, by at least 50%, by at least 60%, by at least 70%, by at least 80%, by at least 90%, by at least 95%, and of course also by 100% as compared to a non-inhibited control cell may be sufficient. A control cell is identical to the inhibited cell safe said inhibition of Menin or the respective other factors. Comparison with the control is at the same environmental conditions (e.g. nutrition, temperature). Suitable Menin inhibitors (or inhibitors of the other factors) and a suitable concentration thereof can be easily tested by one skilled in the art in an in vitro binding assay in comparative cells. Concentrations can be adapted to achieve the desired strength of effect.

Target cells of use in the present invention may be primary cells (non-immortalized cells), such as those freshly isolated from an animal, or may be derived from a cell line capable or prolonged proliferation in culture (e.g., for longer than 3 months) or indefinite proliferation (immortalized cells). Adult somatic cells may be obtained from individuals, e.g. human subjects, and cultured according to standard cell culture protocols available to those of ordinary skill in the art. The cells may be maintained in cell culture following their isolation from a subject. In certain embodiments the cells are passaged once or more following their isolation from the individual (e.g., between 2-5, 5-10, 10-20, 20-50, 50-100 times, or more) prior to their use in a method of the invention. They may be frozen and subsequently thawed prior to use. In some embodiments the cells will have been passaged no more than 1, 2, 5, 10, 20, or 50 times following their isolation from the individual prior to their use in a method of the invention. Passaging may be subculturing by re-cultivation every 3-5 days. In some embodiments, methods of the invention utilize cells of a cell line, e.g., a population of largely or substantially identical cells that have typically been derived from a single ancestor cell or from a defined and/or substantially identical population of ancestor cells or from a tissue sample obtained from a particular individual. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

A preferred embodiment of the invention is wherein the target cells are somatic mammalian cells, preferably, human cells, non-human primate cells, or mouse cells. Preferably, the somatic mammalian cells are fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells.

The cells to be reprogrammed according to the invention are usually somatic cells. Somatic cells of use in the present invention are typically mammalian cells, such as, for example, human cells, non-human primate cells, or mouse cells. They may be obtained by well-known methods from various organs, e.g., skin, lung, pancreas, liver, stomach, intestine, heart, reproductive organs, bladder, kidney, urethra and other urinary organs, etc., generally from any organ or tissue containing live somatic cells. Mammalian somatic cells useful in various embodiments of the present invention may be fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells, skeletal muscle cells, etc., generally any nucleated living somatic cells. In some embodiments, the somatic cell is a terminally differentiated cell, i.e., the cell is fully differentiated and does not (under normal conditions in the body) give rise to more specialized cells. In some embodiments the somatic cell is a terminally differentiated cell that does not divide under normal conditions in the body, i.e., the cell cannot self-renew. In some embodiments, the somatic cell is a precursor cell, i.e., the cell is not fully differentiated and is capable of giving rise to cells that are more fully differentiated. In some embodiments, cells that can be obtained relatively convenient procedure from a human subject are used (e.g., fibroblasts, keratinocytes, circulating white blood cells).

In the methods of the present invention the population of target cells may, in general, be cultured under standard conditions of temperature, pH and other environmental conditions, e.g. as adherent cells in tissue culture plates at 37° C. in an atmosphere containing 5-10% CO₂. The cells and/or the cell culture medium are appropriately modified to achieve reprogramming as described herein. The cell culture medium contains nutrients that are sufficient to maintain viability and, typically, support proliferation of at least some cell types. The medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Some non-limiting examples are provided herein.

As would be appreciated by the skilled person, the quantity of the agent required to reduce the amount and/or activity of one or more reprogramming roadblock factors as mentioned earlier, can vary depending on the type of target cell used in the method of the invention. Similarly, the length of time the target cells are exposed to the agents stated above can vary depending on the type of target cell used in the method of the invention. The quantities and length of time needed to most effectively promote reprogramming in a particular cell type can be readily identified using the methods disclosed herein and also normal experimental procedures. Also, the most effective type of agents can be identified.

For example, the skilled person can perform a series of experiments using the same target cells, then perform the method of the invention using a varying quantity of the agent that inhibits the expression or activity of Menin (as defined above) for a fixed length of time, and then identify the most effective condition for that target cell type. The same applies to the other reprogramming targets instead of Menin. Similarly, the skilled person can perform a series of experiments using the same target cells, then perform the method of the invention using a varying length of time that the cells are exposed to a fixed quantity of the agents, and then identify the most effective condition for that target cell type. Also, similar experiments can be performed where the promoter sequences used in the transient expression system are changed, so as to identify the most optimal system. Furthermore, different methods of transfection of the target cells with the transient expression vectors can used so as to identify the most optimal protocol for the target cells. As can be appreciated, various routine derivatives of the above approach can be used to best identify the conditions to be applied to a particular target cell type using the claimed method.

In the method of the first aspect of the invention, a population of target cells is cultured in medium suitable for culturing iPS cells while undergoing reprogramming. Exemplary serum-containing iPSC medium is made with 80% DMEM (typically KO DMEM), 20% defined fetal bovine serum (FBS) not heat inactivated, 1% non-essential amino acids, 1 mM L-glutamine, and 0.1 mM [beta]-mercaptoethanol. The medium is filtered and stored at 4° C., e.g., for 2 weeks or less. Serum-free ES medium may be prepared with 80% KO DMEM, 20% serum replacement, 1% non-essential amino acids, 1 mM L-glutamine, and 0.1 mM [beta]-mercaptoethanol and a serum replacement such as Invitrogen Cat. No. 10828-028. The medium is filtered and stored at 4° C. Before combining with the cells used for conditioning, human bFGF can be added to a final concentration of 4 ng/mL. StemPro® hESC SFM (Invitrogen Cat. No. A1000701), a fully defined, serum- and feeder-free medium (SFM) specially formulated for the growth and expansion of human embryonic stem cells, is of use. In some embodiments, iPS cells are reprogrammed to one or more differentiated cell types. The iPS cells may be cultured initially in medium suitable for maintaining ES cells and may be transferred to medium suitable for the desired cell type.

The present invention also provides a method for preparing a population of differentiated cells, comprising preparing a population of iPS cells according to the inventive methods and differentiating the iPS cells using a protocol or factor to form a population of differentiated cells.

The method of the invention is used as part of a reprogramming protocol for the preparation of iPS cells.

“Reprogramming protocol” refers to any treatment or combination of treatments that causes at least some cells to become reprogrammed. In some embodiments, “reprogramming protocol” can refer to a variation of a known reprogramming protocol, wherein a factor or other agent used in a known reprogramming protocol is omitted or modified. In some embodiments, “reprogramming protocol” can refer to a variation of a known reprogramming protocol, wherein a factor or agent known to be of use for reprogramming is used together with a different agent whose utility in reprogramming has not been established. Details of reprogramming protocols are now provided below.

In preferred embodiments of the invention, the method comprises (i) expressing one or more Yamanaka factors selected from Oct3/4, Sox2, Klf4, Myc, Nanog and Lin28, and (ii) reducing the amount and/or activity of Menin (Men1), together. “Together” includes the meaning that the expression of the one or more Yamanaka factors and the reduction of amount or activity of Menin act together in a cell with an overlap. E.g. agents that cause the expression and reduction, such as transgenes or inhibitors of Menin act at the same time in the cell but can of course—depending on retention time in the cell—be administered to the cell at different times. For example, one or more Yamanaka factors can be expressed followed by reducing the amount and/or activity of Menin or having the reduced the amount and/or activity of Menin beforehand followed by expression of the one or more Yamanaka factors.

To reprogram somatic cells to pluripotency, the cells may be treated to cause them to express or contain one or more reprogramming factor or pluripotency factor at levels greater than would be the case in the absence of such treatment. For example, somatic cells may be genetically engineered to express one or more genes encoding one or more such factor(s) and/or may be treated with agent(s) that increase the expression of one or more endogenous genes encoding such factors and/or stabilize such factor(s). The agent could be, for example, a small molecule, a nucleic acid, a polypeptide, etc. In some embodiments, pluripotency factors are introduced into somatic cells, e.g. by microinjection or by contacting the cells with the factors under conditions in which the factors are taken up by the cells. In some embodiments the factors are modified to incorporate a protein transduction domain. In some embodiments the cells are permeabilized or otherwise treated to increase their uptake of the factors. The pluripotency factors may also be introduced by ways of integrative or non-integrative nucleic acid approaches. Integrative delivery methods result in the integration of genetic material into the genome of the target cell. Exemplary factors are discussed below.

The transcription factor Oct4 (also called Pou5f1, Oct-3, Oct3/4) is an example of a pluripotency factor. Oct4 has been shown to be required for establishing and maintaining the undifferentiated phenotype of ES cells and plays a major role in determining early events in embryogenesis and cellular differentiation (Nichols et al., 1998, Cell 95:379-391; Niwa et al., 2000, Nature Genet. 24:372-376). Oct4 expression is down-regulated as stem cells differentiate into more specialized cells. Nanog is another example of a pluripotency factor. Oct4 is one of the most preferred Yamanaka factors of the present invention and can be used alone or in combination with any other Yamanaka factor in any embodiment or aspect of the present invention, including methods and kits. Nanog is a homeobox-containing transcription factor with an essential function in maintaining the pluripotent cells of the inner cell mass and in the derivation of ES cells from these. Furthermore, overexpression of Nanog is capable of maintaining the pluripotency and self-renewing characteristics of ESCs under what normally would be differentiation-inducing culture conditions. (See Chambers et al., 2003, Cell 113: 643-655; Mitsui et al., Cell. 2003, 1 13(5):631-42). Sox2, another pluripotency factor, is an HMG domain-containing transcription factor known to be essential for normal pluripotent cell development and maintenance (Avilion, A., et al., Genes Dev. 17, 126-140, 2003). Klf4 is a Krüppel-type zinc finger transcription factor initially identified as a KIf family member expressed in the gut (Shields, J. M, et al., J. Biol. Chem. 271:20009-20017, 1996). Overexpression of Klf4 in mouse ES cells was found to prevent differentiation in embryoid bodies formed in suspension culture, suggesting that Kl f4 contributes to ES self-renewal (Li, Y., et al., Blood 105:635-637, 2005). Sox2 is a member of the family of SOX (sex determining region Y-box) transcription factors and is important for maintaining ES cell self-renewal. c-Myc or “Myc” is a transcription factor that plays a myriad of roles in normal development and physiology as well as being an oncogene whose dysregulated expression or mutation is implicated in various types of cancer (reviewed in Pelengaris S, Khan M., Arch Biochem Biophys. 416(2):129-36, 2003; Cole M D, Nikiforov M A, Curr Top Microbiol Immunol, 302:33-50, 2006). Instead or in addition to expressing Myc, it is also possible to express Lin41 (Trim71), which can replace or enhance Myc activity (Rand et al., Cell Reports 23, 361-375, 2018, incorporated herein by reference). Also, instead or in addition to expressing Myc, it is possible to reduce the expression or activity of p21. These alternatives to Myc can be used in any aspect or embodiment of the invention, including methods and kits. In some embodiments, such factors are selected from the group consisting of: Oct4, Sox2, Klf4, and combinations thereof. In some embodiments a different, functionally overlapping KIf family member such as Klf2 is substituted for Klf4. In some embodiments, the factors include at least Oct4. In some embodiments, the factors include at least Oct4 and a KIf family member, e.g., Klf2. Lin28 is a developmentally regulated RNA binding protein. In some embodiments, somatic cells are treated so that they express or contain one or more reprogramming factors selected from the group consisting of: Oct4, Sox2, Klf4, Nanog, Lin28, and combinations thereof. CCAAT/enhancer-binding-protein-alpha (C/EBPalpha) is another protein that promotes reprogramming at least in certain cell types, e.g., lymphoid cells such as B-lineage cells, is considered a reprogramming factor for such cell types. As an alternative to the Yamanaka factors, any other iPS cell reprogramming factor or combination of iPS cell reprogramming factors can be used. “iPS cell reprogramming factor” are generally factors, similar to Yamanaka factors, that can reprogram or dedifferentiate a somatic cell to an iPS cell. Reducing the amount and/or activity of Menin can also expedite dedifferentiation of such “iPS cell reprogramming factor”. These alternatives to Yamanaka factors can be used in any aspect or embodiment of the invention, including methods and kits.

Accordingly, a preferred embodiment of the present invention relates to a method of preparing a population of iPS cells wherein the step of expressing one or more Yamanaka factors or of treating a cell with a differentiation factor (see below, transdifferentiation in particular), respectively, comprises integrative approaches, preferably retroviral, lentiviral or adenoviral expression vectors, especially excisable and inducible vectors, or non-integrative approaches, preferably integration-defective viral, episomal, RNA or protein delivery techniques, preferably nonviral vector-based IVT-mRNA nanodelivery systems. Especially preferred is the embodiment of the inventive method wherein the integrative or non-integrative approach for expressing one or more Yamanaka factors is transient or inducible.

In one embodiment, the exogenously introduced gene may be expressed from a chromosomal locus other than the chromosomal locus of an endogenous gene whose function is associated with pluripotency. Such a chromosomal locus may be a locus with open chromatin structure, and contains gene(s) whose expression is not required in somatic cells, e.g. the chromosomal locus contains gene(s) whose disruption will not cause cells to die. Exemplary chromosomal loci include, for example, the mouse ROSA 26 locus and type II collagen (Col2al) locus (See Zambrowicz et al., 1997).

Methods for expressing genes in cells are known in the art. Generally, a sequence encoding a polypeptide or functional RNA such as an RNAi agent is operably linked to appropriate regulatory sequences (e.g., promoters, enhancers and/or other expression control elements). Exemplary regulatory sequences are described in Goeddel; Gene Expression Technology: Methods in Enzymology, Academic Press, San Diego, Calif. (1990) [0086]. The gene may be expressed from an inducible or repressible, hence conditional, regulatory sequence such that its expression can be regulated. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotics. A tetracycline-inducible promoter is an example of an inducible promoter that responds to an antibiotic (tetracycline or an analog thereof). See Gossen, M. and Bujard, H., Annu Rev Genet. Vol. 36: 153-173 2002 and references therein. Tetracycline analog includes any compound that displays structural similarity with tetracycline and is capable of activating a tetracycline-inducible promoter. Exemplary tetracycline analogs include, for example, doxycycline, chlortetracycline and anhydrotetracycline.

In some embodiments of the invention expression of an introduced gene, e.g. a gene encoding a reprogramming factor or RNAi agent is transient. Transient expression can be achieved by transient transfection or by expression from a regulatable promoter. In some embodiments expression can be regulated by, or is dependent on, expression of a site-specific recombinase. Recombinase systems include the Cre-Lox and Flp-Frt systems, among others (Gossen, M. and Bujard, H., 2002). In some embodiments a recombinase is used to turn on expression by removing a stopper sequence that would otherwise separate the coding sequence from expression control sequences. In some embodiments a recombinase is used to excise at least a portion of a gene after reprogramming has been induced. In some embodiments the recombinase is expressed transiently, e.g. it becomes undetectable after about 1-2 days, 2-7 days, 1-2 weeks, etc. In some embodiments the recombinase is introduced from external sources.

It is contemplated that protein reprogramming factors, e.g. Yamanaka factors (Oct4, Sox2, Klf4, etc.) may be introduced into cells, thereby avoiding introducing exogenous genetic material. Such proteins may be modified to include a protein transduction domain. Such uptake-enhancing amino acid sequences are found, e.g., in HIV-I TAT protein, the herpes simplex virus 1 (HSV-I) DNA-binding protein VP22, the Drosophila Antennapedia (Antp) transcription factor, etc. Artificial sequences are also of use (see, e.g., Fischer et al, Bioconjugate Chem., Vol. 12, No. 6, 2001 and U.S. Pat. No. 6,835,810).

It is contemplated that a variety of additional agents may be of use to enhance reprogramming. Such agents may be used in combination with an agent that reduces the amount and/or activity of one or more reprogramming roadblock factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and Trp53, especially Menin.

While the present disclosure has focused on reprogramming somatic cells to pluripotency, the inventive methods may be applied to reprogram differentiated somatic or stem cells from a first cell type to a second cell type. For example, it is contemplated that modulating genes and processes identified herein will enhance reprogramming protocols that involve expressing particular combinations of transcription factors in cells to convert them into cells of a different type. Such reprogramming protocols involving modulation of targets identified herein. Differentiation of somatic cells from a first cell type to a second cell type (which is different from the first cell type) is also referred to herein as “transdifferentiation”. As shown herein, inhibition of menin (or of the other reprogramming factors) also enhances transdifferentiation efficiency. The same as said herein for induced stem cell generation in the entire description also applies to transdifferentiation, with the difference that the Yamanaka factors are not required but a differentiation factor for differentiation to the second cell type is used. Accordingly, the invention provides a method of differentiating a cell of a first cell type into a cell of a second cell type that is different from the first cell type comprising treating the cell of the first cell type with a (i) differentiation factor of the second cell type and (ii) reducing the amount and/or activity of Menin (Men1) or one of the other reprogramming factors such as Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and Trp53. The same as said above for reducing the amount and/or activity of Menin (Men1) or one of the other reprogramming factors applies. In other words, the present invention also provides a method of enhanced differentiation of a first cell into a somatic cell of a tissue of interest, comprising (i) treating a cell with a differentiation factor of said tissue of interest, and (ii) reducing the amount and/or activity of Menin (Men1) in a population of target cells. Preferably, (A) said first cell is a cell of low transdifferentiation capacity selected from an adult or mature dermal cell, a blood cell, a hair follicle cell or a urinary cell; or (B) said differentiation is to a somatic cell of a different germ layer than the first cell; or (C) said somatic cell is a non-cardiac cell, preferably also a non-mesoderm-lineage cell (e.g. an endoderm or ectoderm lineage cells).

“First” and “second” are used herein only for distinguishing purposes of the cell types. These first cell type may be selected from hematopoietic stem cells, muscle cells, cardiac muscle cells, liver cells, pancreatic cells, cartilage cells, epithelial cells, urinary tract cells, nervous system cells (e.g., neurons), fibroblasts, adult stem cells, embryonal stem cells, Sertoli cells, granulosa cells, neurons, pancreatic islet cells (also not pancreatic islet cells), epidermal cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells, etc. The second cell type (also referred to as somatic cell of a tissue of interest) may be selected from hematopoietic stem cells, muscle cells, cardiac muscle cells (also not cardiac muscle cells/cardiomyocytes), liver cells, pancreatic cells, cartilage cells, epithelial cells, urinary tract cells, nervous system cells (e.g., neurons), fibroblasts, adult stem cells, embryonal stem cells, Sertoli cells, granulosa cells, neurons, pancreatic islet cells (also not pancreatic islet cells), epidermal cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells, etc.

Transdifferentiation is known in the art, such as disclosed in Ieda et al. Cell Stem Cell 7(2), 2010: 139-141; Liu et al., Cell Division (2016) 2, 16036; Terai et al., J. Biochem. 134, 551-558 (2003); Lázaro et al. Stem Cell Rev and Rep (2016) 12:129-139 (all incorporated herein by reference). Suitable differentiation factors are disclosed in such references and can be used according to the invention. The transcription factor can be for any of the above second cell types, i.e. it mediates differentiation into these cell types.

Preferred transdifferentiations are of bone marrow cells into hepatoblasts or hepatocytes (Terai et al.); of fibroblasts into pluripotent stem cells or cardiomyocytes (Ieda et al. and Liu et al.); of astrocytes or of fibroblasts to neurons; of astrocytes to neuroblasts; of glial cells to neurons; of callosal neurons to cortigofugal neurons; of L4 neurons to L5 neurons; of exocrine cells to beta cells; of fibroblasts to skeletal myofibers or to cardiomycytes; or cardiomyocytes to pacemaker cells (Lázaro et al.); to neuron differentiation (Zhang et al., Neuron 78, 2013: 785-798); of liver cells to insulin-secreting cells (Lazaro et al., US2006/122104 and WO2016/108237); of nonpancreatic cells to pancreatic cells (WO2004/087885); of epidermal cells into neural progenitor cells, neuronal cells and/or glial cells (U.S. Pat. No. 6,949,380). Further documents on various transdifferentiations are WO2005/100550, WO01/95861, EP1642965A, WO01/08691, WO03/066856, WO2006/096640, WO2013/188748, WO2015/010417, WO2015/133879, WO2015/133792, WO2015/131797, WO2016/002937, WO2017/131353. Of course, any other starting (first) cell can be used since differentiation factors determine the target (second, somatic) cell.

The differentiation factor is preferably not of the first cell type, i.e. it is for transdifferentiation. Preferred differentiation factors are selected from Ascl1, Brn2a, Myt1l or Ngn2, neuroD1, Fezf2 (for neuron differentiation); Sox2 (for neuroblast differentiation); from Gata4, Mef2c, Tbx5, Hand2, miRNA 1, mirNA 133, miRNA 208, miRNA 499, (for cardiomyocyte differentiation); Pdx1, Ngn3, MafA (for beta cells); from Pdx1, NeuroD, 6-cellulin, VP16, Ngn3, MafA (for insulin-secreting cells); MyoD, (for skeletal myofibers); Tbx18 (for pacemaker cells); from Brn2, Ascl1, MytL1, Ngn2, NeuroD (for neuron differentiation); such differentiation factors for use alone or in combination are known in the art and reviewed by Lázaro et al. and can be used according to the invention.

Depending on the second or somatic cell of interest, said produced cell can be used in therapy of an (established) disease or condition or for its risk reduction in a prophylactic therapy, such as in the treatment of diabetes (insulin producing cell), heart diseases, cardiovascular disease, ischemia (cardiomyocytes, pacemaker cells), liver insufficiency (liver cells, hepatocytes), anaemia (blood cells), white blood cell insufficiency, e.g. in leukaemia (leukocytes); Alzheimer's diseases, Parkinson's disease, multiple sclerosis (neurons), atrophy (muscle cells, including skeletal muscle cells).

Liu et al., Cell Division (2016) 2, 16036 discloses a transdifferentiation of mouse embryonic fibroblasts into induced cardiomyoctes. Mainly, this document relates to activity of Mll1 H3K4 methyltransferase in order to reprogram cells but also mentions Men1. The teaching of this document is limited to reprogramming of close relatives of cells (both fibroblasts and cardiomyocytes are of mesodermal lineage), starting from easily transdifferentiatable embryonic cells that are not fully mature and lacks the ground-breaking insight provided by the invention that allows complete dedifferentiation to stem cells and any transdifferentiation passing a stem cell-like stage including a transdifferentiation beyond germ layer boundaries. Liu's insights do not form part of the invention and are disclaimed, e.g. in either option A-C mentioned above. Lu et al., Gastroenterology 2010 138: 1954-1965 discloses introducing an insulin-producing activity in pancreatic alpha cells of mesodermal lineage. No transdifferentiating to non-alpha-cells is disclosed and no treatment with differentiation factors. Anyways, in some embodiments of the invention, pancreatic cells are not the (starting) first cells and/or not the second cells or somatic cells of interest.

The invention has shown that transdifferentiation can be facilitated using cells that are usually hard to transdifferentiate or that resist transdifferentiation. Such cells with low transdifferentiation capacity are for example an adult or mature dermal cell, a blood cell, a hair follicle cell or a urinary cell. Adult or mature dermal cells are for example dermal fibroblast (non-embryonic but mature). Such cells can be obtained from a patient, e.g. in a skin sample, and treated according to the invention, both to form iPS cells or to form other somatic cells. Blood cells comprising a nucleus and capable of de- or transdifferentiation are e.g. erythroblasts, myeloblasts, NK cells, lymphocytes, basophils, neutrophils, eosinophils, monocytes, NK cells. Furthermore, hair follicle cells or urinary cells, i.e. cells found in urine, may be used. All these cells are usually hard to de- or transdifferentiate and in some cell donors may not de- or transdifferentiate at all according to prior methods. The inventive menin inhibition may remove such a blockade and allows de- or transdifferentiatiation thereof.

The invention, as said, allows substantial dedifferentiation even to stem cell status. Associated with this dedifferentiation capacity, menin inhibition also allows transdifferentiation across diverse cell types, including between cells of different germ layer lineage. Germ layers are mesoderm, endoderm and ectoderm and all cells have a lineage stemming from these germ layers. Cardiomyocytes, fibroblasts are both mesodermal cells. Neural cells are from ectodermal lineage, in particular neuroectoderm. The invention surprisingly allows transdifferentiation (even without Yamanaka factors but of course use of them is possible) from a cell of a first germ layer to a cell of a different germ layer, different from the first germ layer, such as from a mesodermal-lineage cell to an ectodermal-lineage cell, like a neural cell. Also, the invention provides for the first time a transdifferentiation of cells within the group of ectodermal and/or endodermal cells, including first and second (somatic cell of interest) cells both being ectodermal; or both being endodermal; or between endodermal and ectodermal lineages in any direction. This vast and broad suitability across any cell lineages was unknown and unexpected prior to the invention.

In preferred embodiments of all aspects of the invention, the cells are cultured on or in the presence of a material that mimics one or more features of the extracellular matrix or comprises one or more extracellular matrix or basement membrane components. In some embodiments Matrigel™ is used. Other materials include proteins or mixtures thereof such as gelatin, collagen, fibronectin, etc. In certain embodiments of the invention the ceils are cultured in the presence of a feeder layer of cells. Such cells may, for example, be of murine or human origin. They may be irradiated, chemically inactivated by treatment with a chemical inactivator such as mitomycin c, or otherwise treated to inhibit their proliferation if desired. In other embodiments the target cells are cultured without feeder cells.

The IPS ceils prepared according to the method of the first aspect of the invention may be assessed for one or more characteristics of a desired cell state or cell type. For example, cells may be assessed for pluripotency characteristic(s). The presence of pluripotency characteristic(s) indicates that the target ceils have been reprogrammed to a pluripotent state.

The term “pluripotency characteristics”, as used herein, refers to characteristics associated with and indicative of pluripotency, including, for example, the ability to differentiate into cells derived from all three embryonic germ layers all types and a gene expression pattern distinct for a pluripotent cell, including expression of pluripotency factors and expression of other ES cell markers.

To assess potentially reprogrammed target cells for pluripotency characteristics, one may analyze such cells for particular growth characteristics and ES cell-like morphology. Cells may be injected subcutaneously into immunocompromised SCID mice to determine whether they induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another pluripotency characteristic that can be monitored. One may carry out functional assays of the reprogrammed target cells by introducing them into blastocysts and determining whether the cells are capable of giving rise to all cell types (see Hogan et al., 2003). If the reprogrammed cells are capable of forming SL few cell types of the body, they are multipotent; if the reprogrammed cells are capable of forming all cell types of the body including germ cells, they are pluripotent.

One may also examine the expression of an individual pluripotency factor. Additionally or alternately, one may assess expression of other ES cell markers such as stage-specific embryonic antigens-1, -3, and -4 (SSEA-I, SSEA-3, SSEA-4), which are glycoproteins specifically expressed in early embryonic development and are markers for ES cells (Solter and Knowles, 1978, Proc. Natl. Acad. Sci. USA. 75:5565-5569; Kannagi et al., 1983, EMBO J 2:2355-2361). Elevated expression of the enzyme alkaline phosphatase (AP) is another marker associated with undifferentiated embryonic stem cells (Wobus et al., 1984, Exp. Cell 152:212-219; Pease et al., 1990, Dev. Biol. 141:322-352). Additional ES cell markers are described in Ginis, L, et al., Dev. Biol, 269: 369-380, 2004 and in The International Stem. Cell Initiative, Adewumi O, et al., Nat Biotechnol., 25(7):803-16, 2007 and references therein. For example, TRA-1-60, TRA-1-81, GCTM2 and GCT343, and the protein antigens CD9, Thyl (CD90), class 1 HLA, NANOG, TDGFl, DNMT3B, GABRB3 and GDF3, REX-1, TERT, UTF-1, TRF-I, TRF-2, connexin43, connexin45, Foxd3, FGFR-4, ABCG-2, and Glut-1 are of use.

One may perform, expression profiling of the reprogrammed target cells to assess their pluripotency characteristics. Pluripotent cells, such as embryonic stem cells, and multipotent cells, such as adult stem cells, are known to have a distinct pattern of global gene expression. See, for example, Ramalho-Santos et al., Science 298: 597-600, 2002; Ivanova et al; Science 298: 601-604, 2002; Boyer, L A, et al. Nature 441, 349, 2006, and Bernstein, B E, et al., Cell 125 (2), 315, 2006. One may assess DNA methylation, gene expression, and/or epigenetic state of cellular DNA, and/or developmental potential of the cells, e.g., as described in Wernig, M., et al., Nature, 448:318-24, 2007. Cells that are able to form teratomas containing cells having characteristics of endoderm, mesoderm, and ectoderm when injected into SCID mice and/or possess ability to participate (following injection into murine blastocysts) in formation of chimeras that survive to term are considered pluripotent. Another method of use to assess pluripotency is determining whether the cells have reactivated a silent X chromosome.

Similar methods may be used to assess efficiency of reprogramming cells to a desired cell type or lineage. Expression of markers that are selectively or specifically expressed in such cells may be assessed. For example, markers expressed selectively or specifically by neural, hematopoietic, myogenic, or other cell lineages and differentiated cell types are known, and their expression can be assessed. In some embodiments of the invention the expression level of 2-5, 5-10, 10-25, 25-50, 50-100, 100-250, 250-500, 500-1000, or more RNAs (e.g., mRNAs) or proteins is increased by reprogramming the cell according to the methods of the invention. Functional or morphological characteristics of the cells can be assessed to evaluate the efficiency of reprogramming.

Certain methods of the invention include a step of identifying or selecting cells that express a marker that is expressed by multipotent or pluripotent cells or by cells of a desired cell type or lineage. Standard cell separation methods, e.g., flow cytometry, affinity separation, etc. may be used. Alternately or additionally, one could select cells that do not express markers characteristic of the cells from, which the potentially reprogrammed cells were derived. Other methods of separating cells may utilize differences in average cell size or density that may exist between pluripotent cells and original target cells. For example, cells can be filtered through materials having pores that will allow only certain cells to pass through. Therefore, in some embodiments the present invention provides a method of preparing an IPS cell comprising expressing one or more Yamanaka factors selected from Oct3/4, Sox2, Klf4, Myc, Nanog and Lin28, reducing the amount and/or activity of Menin (Men1) in a population of target cells, and isolating the IPS cell from the target cell population. In another embodiment the present invention provides a method of preparing a population of IPS cells comprising expressing one or more Yamanaka factors selected from Oct3/4, Sox2, Klf4, Myc, Nanog and Lin28, reducing the amount and/or activity of Menin (Men1) in a population of target cells, and isolating the IPS cell population from the target cell population.

In some embodiments the target cells contain a nucleic acid comprising regulatory sequences of a gene encoding a pluripotency factor operably linked to a selectable or detectable marker (e.g., GFP or neo). The nucleic acid sequence encoding the marker may be integrated at the endogenous locus of the gene encoding the pluripotency factor (e.g., Oct4, Nanog) or the construct may comprise regulatory sequences operably linked to the marker. Expression of the marker may be used to select, identify, and/or quantify reprogrammed cells.

Any of the methods of the invention that relate to generating a reprogrammed target cell may include a step of obtaining a target cell or obtaining a population of target cells from an individual in need of cell therapy. IPS cells are generated, selected, or identified from among the obtained cells or cells descended from the obtained cells. Optionally the cell(s) are expanded in culture prior to generating, selecting, or identifying iPS cells genetically matched to the donor.

In some embodiments colonies are subcloned and/or passaged once or more in order to obtain a population of cells enriched for desired cells, i.e iPS cells. The enriched population may contain at least 95%, 96%, 97%, 98%, 99% or more, e.g., 100% cells of a desired type. The invention provides cell lines of target cells that have been stably and heritably reprogrammed to an ES-like state.

In some embodiments, the methods employ morphological criteria to identify reprogrammed cells from among a population of cells that are not reprogrammed to a desired type. In some embodiments, the methods employ morphological criteria to identify target cells that have been reprogrammed to an ES-like state from among a population of cells that are not reprogrammed or are only partly reprogrammed to an ES-like state. “Morphological criteria” is used in a broad sense to refer to any visually detectable feature or characteristic of the cells or colonies. Morphological criteria include, e.g., the shape of the colonies, the sharpness of colony boundaries, the density, small size, and rounded shape of the cells relative to non-reprogrammed cells, etc. For example, dense colonies composed of small, rounded cells, and having sharp colony boundaries are characteristic of ES and iPS cells. The invention encompasses identifying and, optionally, isolating colonies (or cells from colonies) wherein the colonies display one or more characteristics of a desired cell type. The iPS cells may be identified as colonies growing in a first cell culture dish (which term refers to any vessel, plate, dish, receptacle, container, etc, in which living cells can be maintained in vitro) and the colonies, or portions thereof, transferred to a second cell culture dish, thereby isolating reprogrammed cells. The cells may then be further expanded.

The present invention provides IPS cells produced by the methods of the invention. These cells have numerous applications in medicine, agriculture, and other areas of interest. The invention provides methods for the treatment or prevention of a condition in SL mammal. In one embodiment, the methods involve obtaining somatic cells from the individual, using these to prepare a target cell population, and preparing a population of iPS cells according to the claimed invention.

In certain embodiments of the invention the obtained iPS cells are then cultured under conditions suitable for their development into cells of a desired cell type, i.e. they then become re-differentiated iPS cells. The cells of the desired cell type are introduced into the individual to treat the condition. The IPS cells can also be induced to develop a desired organ, which is harvested and introduced into the individual to treat the condition. The condition may be any condition in which cell or organ function is abnormal and/or reduced below normal levels. Thus, the invention encompasses obtaining somatic cells from, an individual in need of cell therapy, using these cells as the target cell population in the claimed method, and optionally differentiating IPS cells to generate cells of one or more desired cell types, and introducing the cells into the individual. An individual in need of cell therapy may suffer from any condition, wherein the condition or one or more symptoms of the condition can be alleviated by administering cells to the donor and/or in which the progression of the condition can be slowed by administering cells to the individual. The method may include a step of identifying or selecting reprogrammed somatic cells and separating them from cells that are not reprogrammed.

The IPS cells, and thus may be induced to differentiate to obtain the desired cell types according to known methods to differentiate such cells. For example, the IPS cells may be induced to differentiate into hematopoietic stem cells, muscle cells, cardiac muscle cells, liver cells, pancreatic cells, cartilage cells, epithelial cells, urinary tract cells, nervous system cells (e.g., neurons) etc., by culturing such cells in differentiation medium and under conditions which provide for cell differentiation, Medium and methods which result in the differentiation of embryonic stem cells obtained using traditional methods are known in the art, as are suitable culturing conditions. Such methods and culture conditions may be applied to the IPS cells obtained according to the present invention (see, e.g., Trounson, A., The production and directed differentiation of human embryonic stem cells, Endocr Rev. 27(2): 208-19, 2006 and Yao, S., et al, Long-term self-renewal and directed differentiation of human embryonic stem cells in chemically defined conditions, Proc Natl Acad Sci USA, 103(18): 6907-6912, 2006).

Thus, using known methods and culture media, one skilled in the art may culture IPS cells to obtain desired differentiated cell types, e.g., neural cells, muscle cells, hematopoietic cells, etc. The subject cells may be used to obtain any desired differentiated cell type. Such differentiated human cells afford a multitude of therapeutic opportunities. For example, human hematopoietic stem cells derived from cells reprogrammed according to the present invention may be used in medical treatments requiring bone marrow transplantation. Such procedures are used to treat many diseases, e.g., late stage cancers and malignancies such as leukemia. Such cells are also of use to treat anemia, diseases that compromise the immune system such as AIDS, etc. The methods of the present invention can also be used to treat, prevent, or stabilize a neurological disease such as Alzheimer's disease, Parkinson's disease, Huntington's disease, or ALS, lysosomal storage diseases, multiple sclerosis, or a spinal cord injury. For example, somatic cells may be obtained from the individual in need of treatment, and reprogrammed to gain pluripotency, and cultured to derive neurectoderm cells that may be used to replace or assist the normal function of diseased or damaged tissue. For example, in the course of the present, invention it was found that transdifferentiation of somatic cells to neurons is more efficient when IPS cells are generated according to the inventive method, hence when Menin activity or expression is reduced.

Re-diffentiated iPS cells that produce a growth factor or hormone such as insulin, etc., may be administered to a mammal for the treatment or prevention of endocrine disorders. Re-diffentiated iPS cells that form epithelial cells may be administered to repair damage to the lining of a body cavity or organ, such as a lung, gut, exocrine gland, or urogenital tract. It is also contemplated that iPS may be administered to a mammal to treat damage or deficiency of cells in an organ such as the bladder, brain, esophagus, fallopian tube, heart, intestines, gallbladder, kidney, liver, lung, ovaries, pancreas, prostate, nerves, spinal cord, spleen, stomach, testes, thymus, thyroid, trachea, ureter, urethra, or uterus.

iPS cells may be combined with a matrix to form a tissue or organ in vitro or in vivo that may be used to repair or replace a tissue or organ in a recipient mammal (such methods being encompassed by the term “cell therapy”). For example, iPS cells may be cultured in vitro in the presence of a matrix to produce a tissue or organ of the urogenital, cardiovascular, or musculoskeletal system. Alternatively, a mixture of the cells and a matrix may be administered to a mammal for the formation of the desired tissue in vivo. The iPS cells produced according to the invention may be used to produce genetically engineered or transgenic differentiated cells, e.g., by introducing a desired gene or genes, or removing all or part of an endogenous gene or genes of IPS cells produced according to the invention, and allowing such cells to differentiate into the desired cell type. One method for achieving such modification is by homologous recombination, which technique can be used to insert, delete or modify a gene or genes at a specific site or sites in the genome.

This methodology can be used to replace defective genes or to introduce genes which result in the expression of therapeutically beneficial proteins such as growth factors, hormones, lymphokines, cytokines, enzymes, etc. For example, the gene encoding brain derived growth factor may be introduced into human embryonic or stem-like cells, the cells differentiated into neural cells and the cells transplanted into a Parkinson's patient to retard the loss of neural cells during such disease. Using known methods to introduced desired genes/mutations into IPS cells, the IPS cells may be genetically engineered, and the resulting engineered cells differentiated into desired cell types, e.g., hematopoietic cells, neural cells, pancreatic cells, cartilage cells, etc. Genes which may be introduced into the IPS cells include, for example, epidermal growth factor, basic fibroblast growth factor, glial derived neurotrophic growth factor, insulin-like growth factor (I and II), neurotrophin3, neurotrophin4/5, ciliary neurotrophic factor, AFT-1, cytokine genes (interleukins, interferons, colony stimulating factors, tumor necrosis factors (alpha and beta), etc.), genes encoding therapeutic enzymes, collagen, human serum albumin, etc.

Negative selection systems known in the art can be used for eliminating therapeutic cells from a patient if desired. For example, cells transfected with the thymidine kinase (TK) gene will lead to the production of reprogrammed cells containing the TK gene that also express the TK gene. Such cells may be selectively eliminated at any time from a patient upon gancyclovir administration. Such a negative selection system is described in U.S. Pat. No. 5,698,446. In other embodiments the cells are engineered to contain a gene that encodes a toxic product whose expression is under control of an inducible promoter. Administration of the inducer causes production of the toxic product, leading to death of the cells. Thus, any of the somatic cells of the invention may comprise a suicide gene, optionally contained in an expression cassette, which may be integrated into the genome. The suicide gene is one whose expression would be lethal to cells. Examples include genes encoding diphtheria toxin, cholera toxin, ricin, etc. The suicide gene may be under control of expression control elements that do not direct expression under normal circumstances in the absence of a specific inducing agent or stimulus. However, expression can be induced under appropriate conditions, e.g., (i) by administering an appropriate inducing agent to a cell or organism or (ii) if a particular gene (e.g., an oncogene, a gene involved in the cell division cycle, or a gene indicative of dedifferentiation or loss of differentiation) is expressed in the cells, or (ill) if expression of a gene such as a cell cycle control gene or a gene indicative of differentiation is lost (see, e.g. U.S. Pat. No. 6,761,884). In some embodiments the gene is only expressed following a recombination event mediated by a site-specific recombinase. Such an event may bring the coding sequence into operable association with expression control elements such as a promoter. Expression of the suicide gene may be induced if it is desired to eliminate cells (or their progeny) from the body of a subject after the cells (or their ancestors) have been administered to a subject. For example, if SL reprogrammed somatic cell gives rise to a tumor, the tumor can be eliminated by inducing expression of the suicide gene. In some embodiments tumor formation is inhibited because the cells are automatically eliminated upon dedifferentiation or loss of proper cell cycle control.

Examples of diseases, disorders, or conditions that may be treated or prevented include neurological, endocrine, structural, skeletal, vascular, urinary, digestive, integumentary, blood, immune, auto-immune, inflammatory, endocrine, kidney, bladder, cardiovascular, cancer, circulatory, digestive, hematopoietic, and muscular diseases, disorders, and conditions. In addition, reprogrammed cells may be used for reconstructive applications, such as for repairing or replacing tissues or organs. In some embodiments, it may be advantageous to include growth factors and proteins or other agents that promote angiogenesis. Alternatively, the formation of tissues can be effected totally in vitro, with appropriate culture media and conditions, growth factors, and biodegradable polymer matrices.

The present invention contemplates all modes of administration, including intramuscular, intravenous, intraarticular, intralesional, subcutaneous, or any other route sufficient to provide a dose adequate to prevent or treat a disease. The iPS cells may be administered to the mammal in a single dose or multiple doses. When multiple doses are administered, the doses may be separated from one another by, for example, one week, one month, one year, or ten years. One or more growth factors, hormones, interleukins, cytokines, or other cells may also be administered before, during, or after administration of the cells to further bias them, towards a particular cell type.

The iPS cells obtained using methods of the present invention are unique in that the amount and/or activity of Menin is reduced. Accordingly, a further aspect of the present invention is to provide a population of iPS cells prepared according to any of the inventive methods wherein the amount and/or activity of Menin is reduced compared to iPS cells that have not been treated with a Menin-reducing agent.

The iPS cells according to the present invention may be used as an in vitro model of differentiation, e.g., for the study of genes which are involved in the regulation of early development. Differentiated cell tissues and organs generated using the reprogrammed cells may be used to study effects of drugs and/or identify potentially useful pharmaceutical agents. In general the iPS cells of the present invention may also be used for any of the above-described therapeutic approaches.

The reprogramming methods disclosed herein may be used to generate iPS cells, for a variety of animal species. The iPS cells generated can be useful to produce desired animals. Animals include, for example, avians and mammals as well as any animal that is an endangered species. Exemplary birds include domesticated birds (e.g., chickens, ducks, geese, turkeys). Exemplary mammals include murine, caprine, ovine, bovine, porcine, canine, feline and non-human primate. Of these, preferred members include domesticated animals, including, for examples, cattle, pigs, horses, cows, rabbits, guinea pigs, sheep, and goats. Preferred cells are however human cells to be used in the inventive method.

Hence a further aspect of the invention provides a method for preparing a population of differentiated cells, comprising (i) preparing a population of IPS cells according to the method of the first aspect of the invention, (ii) differentiating the iPS cells using a protocol or factor to form a population of differentiated cells. Methods for reprogramming the cells and their utility are provided above.

A further aspect of the invention provides a population of iPS cells prepared according to the method of the first aspect of the invention. In one embodiment of the invention the iPS cell population according to the invention has reduced amount and/or activity of Menin. In another embodiment the inventive iPS cell population according to the present invention has reduced amount and/or activity of one or more of the following reprogramming roadblock factors identified by the inventors: Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and Trp53.

A further aspect of the invention provides cell culture media comprising one or more agents that inhibit the expression, translation or activity of Menin. Cell culture media according to the present invention may also comprise one or more agents that inhibit the expression, translation or activity of Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53. In some embodiments of the present invention the cell culture medium comprises only one or more agents that inhibit the expression, translation or activity of Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1 Pten, Senp1 and/or Trp53, but not Menin-inhibiting agents.

In some embodiments the invention also provides cell culture media comprising Yamanaka factors or Yamanaka-inducing agents and one or more agents that inhibit the expression or activity of Menin. In another embodiment the invention provides cell culture media comprising Yamanaka factors or Yamanaka-inducing agents and one or more agents that inhibit the expression or activity of one or more reprogramming roadblock factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53.

Another aspect of the present invention provides a kit for enhanced reprogramming of somatic cells into IPS cells. The kit according to the present invention comprises one or more cell culture media used to generate IPS ceils from somatic cells. Preferably, the cell culture media provided in the kit according to the present invention comprises Sendai virus vectors encoding one or more reprogramming roadblock factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53. More preferably, the cell culture media provided in the kit comprises one or more Sendai virus vectors encoding Yamanaka factors and one or more Sendai virus vectors encoding reprogramming roadblock factors selected from Menin, Socs3, Eif4e2, Apc, Setd2, Axin1, Cdk13, Psip1, Cabin, Fbxw7, Tcf711, Tlk2, Hira, Uba2, Ubr4, Sae1, Chaf1a, Asf1a, Dot1l, Pias1, Pten, Senp1 and/or Trp53. The kit according to the present invention may further comprise cell culture media for re-differentiating the generated IPS cells into differentiated cells. Preferably, the cell culture media used for re-differentiating comprises agents that direct differentiation towards neurons.

The term “Yamanaka factors” comprises all standard reprogramming factors as described above, for example Oct3/4, Sox2, Klf4, Myc, Nanog and Lin28.

The present invention is further explained by way of the following examples and the figures. The examples are for illustrative purposes only and are not intended to limit the scope of the present invention.

### FIGURES

FIG. 1: Schematic illustration of UMI use in CRISPR/Cas9 screens. Data analysis in CRISPR screens is conventionally based on several sgRNAs targeting the same gene. Introduction of random barcodes at complexities well above total analyzed cell number will tag each individual cell with a unique molecular identifier (UMI). This generates a third layer of information at single cell level, that represents true biological replica.

FIG. 2: Conceptual advantage of CRISPR screen analysis by single cell tracing, (a) Upon infection with sgRNA libraries and selection, each infected cell gives rise to cell colonies of daughter cells with various editing outcomes. Here only 1 guide infecting independent cells is shown. Homozygous frameshifts resulting in loss of function alleles (LOF) are shown in solid color, for alternative editing outcomes only the nucleus is labelled. Negative selection results in depletion of LOF cells, (b) Conventional CRISPR screen analysis by NGS will only detect partial depletion of sgRNA reads (red lines) masking the biological component of the effect by technical limitations (e.g. guide efficiency, LOF frequency), thus limiting the possible depletion level (c) Upon limiting dilution and clonal expansion, each infected cell will be tagged with a unique tag (UMI) and represent a clonal editing outcome, whereby LOF clones show complete negative selection (d) Single cell based CRISPR analysis using NGS scores depletion in LOF clones based on biological phenotype, depletion level is approximated by median depletion of clones. (e) Positive selection of a guide can be due to high penetrance or high degree of outgrowth, (f) unlike conventional analysis, CRISPR-UMI analysis can distinguish stochasticity and quantity of effects.

FIG. 3: Bioinformatic pipeline of sgRNA prediction. Doench-scores as measure of predicted sgRNA activity were calculated for all exonic sgRNAs compatible with our cloning strategy. Doench scores were penalized based on a ruleset for biological effects. Those rules combine evaluation of exon length, prediction of protein domains, alternative splicing and ATG start codons, Pol-II terminator sequences, position of the sgRNA within the CDS. The penalties also spread selected sgRNAs over different exons and an off-target prediction penalizes sgRNAs with predicted off-targets.

FIG. 4: Library cloning and sequencing strategy, (a) Vector design for library generation. Upon pooled parallel cloning of unique molecular identifiers (UMI) into retroviral backbones at complexities of 106, chip-synthesized sgRNA pools (at a complexity of 26514) were cloned into UMI containing backbone at a coverage >1000 clones/guide. Cassette-flanking Pad sites allowed for liberation of small sgRNA containing fragments from mammalian genomic DNA; (b) Library subpools and cloning complexity resulting in overall complexity of 83 million, (c) Ethidium bromide stained agarose gel, 200 ng DNA/lane; Digest of genomic DNA after screens and plasmid DNA as control with the octamer recognition site enzyme Pad results in mostly large genomic fragments, while sgRNA fragments are 589 bp long (arrowhead). Long and short fragments can be fractionated using magnetic beads, (d) Genomic Q-PCR on fractions shown in (c); Short target region is enriched 4.1*10³ in fraction 2. Error bars are STDEV, shown is one experiment in technical triplicate representative of 3 experiments. (e) Illumina SR50 NGS sequencing strategy; First read by custom U6 primer, both index reads by standard illumina primers (f) guide representation in sgRNA libraries snows 4-fold representation difference between poorly and highly represented clones (10th and 90th percentile). Reads normalized between individual subpools.

FIG. 5: Scheme illustrating generation of CRISPR-UMI library complexity. The CRISPR-UMI library is generated by 2 subsequent complex cloning steps. Initially, a random barcode consisting of 10 nucleotides is integrated into the vector backbone. Subsequently, the sgRNA pool of 30 000 sgRNAs is ligated to the barcode library with >1000 ligation events/sgRNA. Thereby, each of the >1000 ligation events/sgRNA combines the sgRNA with another random barcode. The combination of sgRNA and random barcodes generates a complexity of >1000 times the number of sgRNAs. We refer to this highly complex combination of sgRNA and barcode as UMI (unique molecular identifier). Our library reached a complexity of 83 million (see also FIG. 4).

FIG. 6: Pilot screen to identify optimal conditions for UMI based CRISPR screen analysis, (a) Setup of screen; Upon editing, various clonal outgrowth regiments, followed by clonal expansion and dropout screening, were run in parallel. Cas9 expression was induced by Dox, selection for cells harboring guide RNAs was performed by G418 selection. Limiting dilution and expansion is variable in the experiment. Cells are treated with or without 3.3 nM etoposide a LD₃₀ for 8 days, (b) Scheme illustrating variation in clone number and size (c) Average clone numbers and size determined from NGS data (d) Distribution of single cell derived clones in each regimen illustrated with guide_1 against Nhej1. P-value for each clone correlates with read depth but results in less data points, (e) Plot illustrating median dropout for each condition as well as p-value determined by combining multiple clones using MAGeCK. Signal to noise ratios (SNR) are highest in 148 clones of 35 reads, and the percentage of guides expected to have less than 5 clones due to variability in representation is with 0.06% lower than for 52 or 21 clone datasets.

FIG. 7: Single cell analysis of negative selection screen. (a) Graphical illustration for large scale screen setup used to identify sensitizing mutations for etoposide. Cas9 expression was induced by Box, selection for cells harboring guide RNAs was performed by G418 selection, (b) Volcano plot of conventional CRISPR analysis; sgRNA representation relative to control on X-axis, binominal p-value on y-axis, (c) volcano plot of single cell derived clones (CRISPR-UMI analysis), axis as in (a), median depletion of each guide is shown as dashed line, (d) Conventional analysis of depleting sgRNAs, 4 sgRNAs/gene discussed below are highlighted in the same color, (d) CRISPR-UMI analysis of same sgRNAs, p-value is based on MAGeCK score of individual clones within the population, depletion level relative to controls and signal to noise ratio is shown below for (d) and (e).

FIG. 8: Comparison of conventional analysis performance with CRISPR-UMI. (a) Comparison of conventional and single cell based analysis on guide level, discrepancies highlighted before (diamond) and after (asterisk) outlier removal, (b) For discrepant clones, abundance relative to untreated control (X-axis) against total reads per clone (Y-axis) shows strong outlier clones dominating total read space. Depleted clones lie on the left side of the plot, (c) Venn Diagrams illustrating the number of sgRNAs targeting the positive controls of the NHEJ complex (Lig4, Xrcc4-6, Nhej1) called within the top 50/100 hits (d) Average number of guides targeting the same gene as function of top number of sgRNAs. CRISPR-UMI shows higher reproducibility/number of guides per gene across the entire range of hits, (e) Top ranking genes based on conventional analysis as well as CRISPR-UMI. Ranking of hits avoids false positive and negative calls and shows stronger depletion in CRISPR-UMI. (f) Clonal validation of selected hits at 10 nM etoposide treatment (5 nM pre-treatment) for 3 days. Homozygous loss of function mutations in Lig4, Zfp451, Rad9a, and Erbb4 show sensitization to etoposide. 4 clones per gene (2 for Trim71 and Rac1), 3 technical replicates each, error bars are SEM; Heteroskedastic, two-sided t.test was applied p<0.01=***, p<0.01=**, p<0.05=*, p>0.05=ns. Number of samples is 12 for Lig4, Zfp451, Slc25a4, Adcy3, Rad9a, Erbb4, empty and wt and 6 for Rac1 and Trim71. (g) Visual summary of all genes identified by CRISPR-UMI. All genes, with the exception of Zfp451, have previously been implicated in DNA damage response or repair as (1) involved in resolution of Topoisomerase II entangled chromosomes, (2) NHEJ, or (3) putatively in microhomology based end joining, or (4) SUMOylation in response to DNA damage (Srivastava, M, et al. Cell 151, 1474-1487 (2012), Kurosawa, A. et al. PLoS ONE 8, e72253 (2013), Fattah, F. J. et al. DNA Repair (Amst.) 15, 39-53 (2014), Jackson, S. P. & Bartek, J. Nature 461, 1071-1078 (2009), Black et al. Genes (Basel) 7, 67 (2016), Takata et al. Nat Commun 4, 2338 (2013), Gilmore-Hebert, M., Ramabhadran, R. & Stern, D. F. Mol. Cancer Res. 8, 1388-1398 (2010), Icli, B., Bharti, A., Pentassuglia, L., Peng, X. & Sawyer, D. B. Biochem. Biophys. Res. Commun. 418, 116-121 (2012), Smilenov, L, B. et al. Cancer Res. 65, 933-938 (2005), Koidl, S. et al. The International Journal of Biochemistry & Cell Biology 79, 478-487 (2016), Guzzo, C. M. et al. Sci Signal 5, ra88-ra88 (2012), Kont, Y. S. et al. DNA Repair (Amst.) 43, 38-47 (2016), Katsube, T. et al. J. Radiat. Res. 52, 415-424 (2011) and Pommier, Y. et al. DNA Repair (Amst.) 19, 114-129 (2014)).

FIG. 9: Read distributions of single cell derived clones. (a) No strong outlier clones are detected in hits identified by conventional analysis as well as CRISPR-UMI (b) Strong outlier clones with very high read counts as well as depletion are seen in false positive hits called by conventional analysis, (c) Genes identified only in CRISPR-UMI show modest but reproducible depletion in multiple independent clones but are often dominated by clones with high read counts that to not deplete.

FIG. 10: Pooled dropout screen without clonal outgrowth also shows outliers resulting in false positive calls, (a) Comparison of CRISPR-UMI with conventional screen analysis on guide level in absence of clonal dilution and outgrowth shows highly correlative results (yellow) as well as discrepancy between both regimen (mixed colors, Pearson correlation: 0.729) (b) While correlating sgRNAs do not contain strong outlier clones based on total read count, guides only called in conventional analysis snow outlier clones responsible for overall dropout, (c) Ranking of sgRNAs improves upon removal of outlier clones (top 3 clones by read count) from the dataset illustrating their confounding effects.

FIG. 11: Single cell analysis of positive selection screen for roadblocks of reprogramming, (a) Schematic of experimental setup. Mouse embryonic fibroblasts carrying T3G-OKSM in the ColA locus, rtTA in the ROSA locus, and a knocking of GFP in the Oct4 locus were infected with lentiviral encoded Cas9 and subsequently with a retroviral library delivering sgRNAs. Reprogramming was induced by Box administration for 7 days, and GFP-positive IPS cells were purified by FACS on day 11. Evaluation of individual IPS colonies by UMIs. (b) Scatterplot shows enrichment of individual sgRNAs based on abundance (total read counts) on the y axis, and enrichment of individual sgRNAs based on incidence (independent colony number) on the x-axis. Both axes are normalized to the total number of reads (abundance) of the sgRNA in the untreated MEF population. Median enrichment of sgRNAs in 4 experiments is shown (c) Single sgRNA validation in 6-well format in triplicate. Readout was by flow cytometry measuring fraction of GFP positive cells. As the validation was done in 4 batches and each batch shows slightly different efficiency, we normalize to controls. Knockdown of Ube2i results in an improvement of reprogramming >100 fold in low dose OKSM. Error bars are STDEV. (d) Alkaline phosphatase staining in 6-wells on day 10 illustrates enhanced reprogramming upon Men1 or Pias1 targeting in the transgenic system, (e) Box plot of colony size, assayed using the normalized and median scaled abundance of unique barcode-guide combinations, revealed similar distributions for all guides targeting one gene and illustrates increased colony size due to faster reprogramming or faster IPS colony growth. (f) Representative colonies in validation experiment on day 10 after Box induction stained for alkaline phosphatase activity confirms colony size predictions.

FIG. 12: Predicted size distribution of colonies by UMI analysis from NGS data, (a) Alkaline phosphatase staining in 6 well dishes 10 days after Box induction in the transgenic system illustrating enhanced IPS colony formation for guides targeting Men1 or Pias1. (b) Median size distribution of read counts per UMI for each sgRNA. Reads for each UMI were filtered for sequencing errors and median colony size is plotted relative to median size illustrating a marked size increase per iPS colony in many but not all identified roadblocks of reprogramming, (c) Representative colonies for comparison with FIG. 8f stained with alkaline phosphatase in validation experiment on day 10 after Box.

FIG. 13: alkaline phosphatase (AP) staining of reprogrammed iPS cells. AP stains iPS cell colonies dark blue (arrowheads), while fibroblasts do not stain or appear as fibroblastic stained cells (asterisk). Reprogramming by sh menin (samples 9 and 10) or sg menin (samples 19 and 20) control is shown in samples 21 and 22,

FIG. 14: Differentiation ESC to iN. Mean number of iN derived from Ascl1 and Ngn2 cell line with and without menin knockout is shown in (a). The boxplots (b) and (c) show data from two clones with confirmed homozygous menin knockout and the corresponding parent cell line without menin knockout. Cell numbers counted using FACS are plotted from three independent experiments N=3, error bars=standard deviation

FIG. 15: Transdifferentiation MEF to iN. (a) and (b): cell images with and without menin knockout. The plot of (c) illustrates the difference in iN number obtained from Ascl1 cell line after menin knockout and empty guide control. N=3, error bars=standard deviation.

### EXAMPLES

### Example 1: Guide Selection

sgRNAs targeting mouse nuclear genes as well as drugged orthologs and a set of hand selected genes with 4 sgRNAs per gene (5 sgRNAs per gene for the subset drugged genes) were selected by a bioinformatics pipeline. We aimed to design a guide selection algorithm taking both guide efficiency as well as biological effect due to gene structure into account. The basis of the guide selection is the activity score as described by Doench et al. (Nature Biotechnology 32, 1262-1267 (2014)). Additionally, we identified properties of each guide and exon under consideration and penalized the Doench score accordingly. We identified all exonic PAM sites in the mouse genome mm10 (Rosenbloom et al. The UCSC Genome Browser database: 2015 update. Nucleic Acids Res. 43, D670-81 (2015)). We excluded sgRNAs that are incompatible with our cloning strategy (contain: GAAGAC, GTCTCC, CTCGAG, CGTCTC or GAGACG, start with: AAGAC or end with: CTCGA). We then calculated Doench-scores for all potential sgRNAs. We penalized the Doench-scores based on heuristic rules (exact penalty scores can be found in FIG. 3) that aim to select sgRNAs which most likely lead to LOF phenotypes. Those rules include exon properties such as presence or absence of protein domains annotated in Pfam database (Finn et al. Nucleic Acids Res. 44, D279-85 (2016)), exon size, and whether or not exon length is a multiple of 3 bp. Then we created penalties for exon distribution, in order to spread sgRNAs over many exons where only the sgRNA with the best Doench score per exon does not get penalized. We also avoided sgRNAs that are less than 4 nt away from another better scoring sgRNA. Furthermore we penalized sgRNAs that cut DNA upstream of a possible alternative ATG start codon and sgRNAs that cut in exons that are not common to all annotated transcripts from that locus. We avoided sgRNAs that contain a stretch of 4 or more T in a row which would act as a Pol-III Terminator. We calculated a distance-penalty based on the distance from the sgRNA to the transcriptional start ranging from 1 to 0.5. Then we calculated a simple off-target prediction (FIG. 3) against all exonic sequences containing a PAM site.

The off-target prediction scores weight mismatches by position in the sgRNA sequence. We re-ranked the penalized Doench score including the off-target analysis and picked the top 4 sgRNAs per gene (the top 5 sgRNAs for Druggable genes) for chip oligo synthesis (CustomArray Inc.). For negative control guides we used a published list of human control guides (Wang et al. Science 343, 80-84 (2014)) and removed all guides which had a perfect match against the mouse genome. We included a total of 112 control guides into our mouse library targeting 6560 genes.

### Example 2: Library Cloning

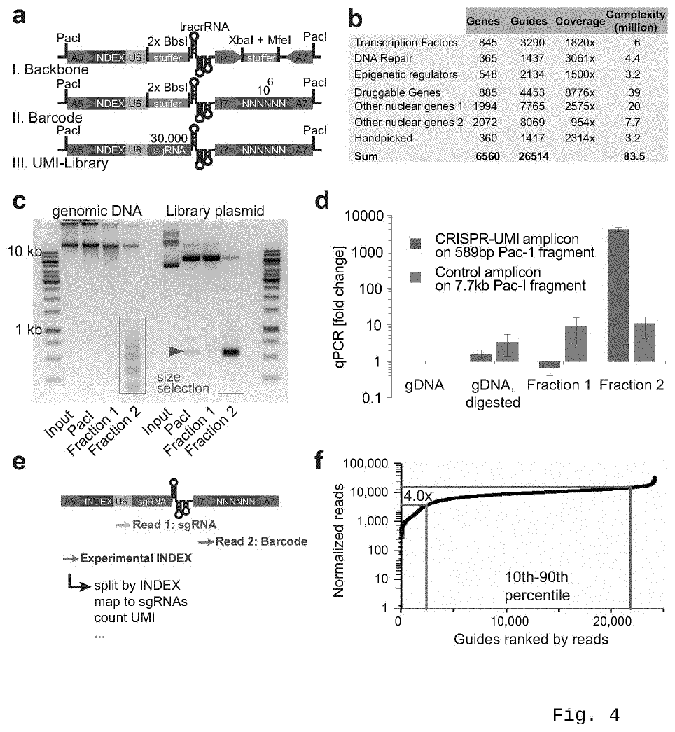

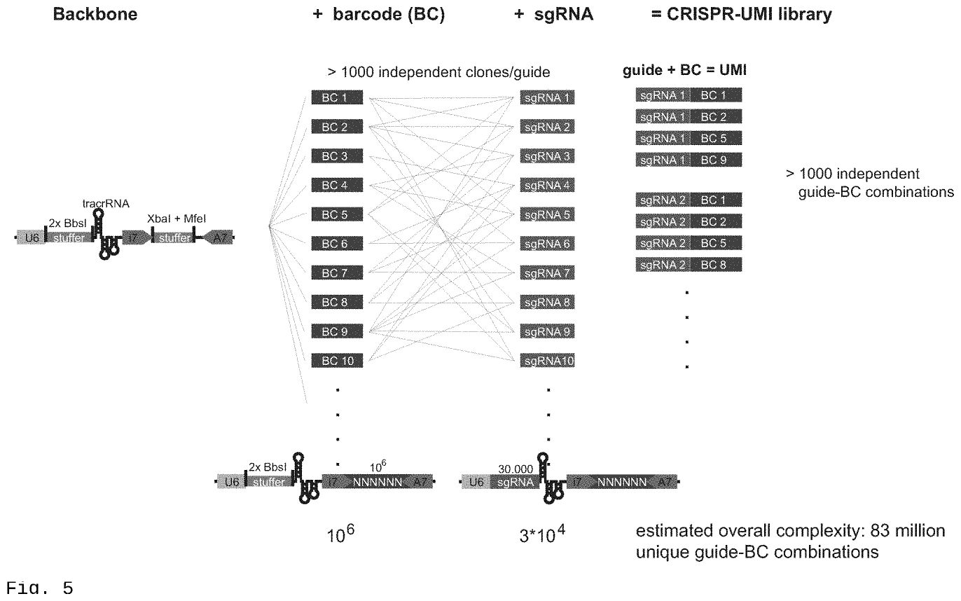

We ordered a gBlock (IDT) flanked by primer binding sites for amplification, restriction sites EcoRI and MfeI for cloning the Illumina i7 primer binding site followed by 10 bp random nucleotide sequence and the Illumina P7 Adaptor. (acgatgagcagagccagaaccagaaggaacttgactctagaGATCGGAAGAG-CACACGTCTGAACTCCAGTCACNNNNNNNNNNgtcctcatctgagagctactcatcaacgg-tATCTCGTATGCCGTCTTaTGCTTGTTAATTAAGAATTCctggacga, SEQ ID NO: 1) (note: we exchanged C to A in the P7 Adaptor Sequence to eliminate a BbS-I restriction site in the adaptor for library cloning, but reintroduced the C during PCR in the DNA-sample prep before NGS). The gBock was digested with EcoRI (NEB R3101L) and MfeI (NEB R3589L) purified on a column (Qiagen 27106) and precipitated with Ethanol. Vector backbone (see FIG. 4a) was digested with XbaI (NEB R0145L) and MfeI (NEB R3589L) and dephosphorylated with rSAP (NEB M0371L), a 1.5 kb stuffer containing a EcoRI restriction site was excised, vector backbone fragments were separated by agarose gel electrophoresis, gel extracted (QIAGEN 28704) and precipitated with Ethanol. 2 μg of vector were ligated with 125 ng plasmid at a molar ration of V:I=1:3, 2 μl T4 DNA Ligase (NEB M0202M) in a total volume of 200 μl split into 10 reactions of 20 μl each. The ligation was purified using a column (Qiagen 27106) and electroporated into electrocompetent XL-1 Blue cells (Agilent 200249) 80 μl in 0.2 cm cuvette 2.5 kV (BioRad, Gene Pulser II), based on colony count the electroporation complexity was estimated to be 1 million. In a second library-cloning step, sgRNAs were cloned into vector containing complex barcodes. The vector was digested using BbsI (NEB R0539L or Fermentas ER1011), excising a stuffer containing an XhoI binding site, dephosphorylated with rSAP (NEB M0371L), linear Fragments isolated from agarose gel electrophoresis, and Ethanol precipitated. sgRNAs were ordered on a chip (CustomArray Inc.) and subsets of the oligos amplified with specific flanking primers with 10 cycles PCR. PCR product was purified on a column (Qiagen 27106) and digested overnight with BbSI (NEB R0539L or Fermentas ER1011) Vector and Insert were ligated in a golden gate reaction using 0.25 μl T4 DNA Ligase (NEB M0202M) and 1 μl BbSI (NEB R0539L or Fermentas ER1011) in 50 μl reaction volume. The reaction was cycled 20 times (5 min 37° C., 5 min 16° C., 20×) followed by 10 min 50° C. inactivation. Plasmids were purified on columns (Qiagen 27106), ethanol precipitated and electroporated into electrocompetent XL-1 Blue cells (Agilent 200249). Electroporated XL-1 Blue cells were collected in 2 ml recovery diluent and incubated at 37 C 200 rpm for 40 min, cells were plated on 2 square 245×245 mm LB agar plates containing 100 μg/ml Ampicillin (Thermo 166508) and incubated at 37 C for 10 h. Bacteria were collected from the plates and grown in 2L LB-Amp at 100 μg/ml (Sigma A9518) for 2 4 h until OD 2.0. Plasmid DNA was prepared (Macherey-Nagel NucleoBond Xtra Maxi Kit) according to manufacturer's recommendations.

### Example 3: ES Cell Culture

A murine embryonic stem cell clone, derived from a derivative of HMSc2 termed AN3-12, with doxycycline inducible Cas9 (T3G-Cas9-IRES-mcherry PGK-GFP-rtTA) was used for this study. The following ES cell medium (ESCM) was used: 450 ml DMEM (Sigma D1152); 75 ml FCS (Invitrogen); 5.5 ml P/S (Sigma P0781); 5.5 ml NEAA (Sigma M7145); 5.5 ml LGlu (Sigma G7513); 5.5 ml NaPyr (Sigma S8636); 0.55 ml beta-mercapto ethanol (Merck 805740; dilute 10 μl bME in 2.85 ml PBS for a 1000× stock), 7.5 μl LIF (IMBA-MolBioService; 2 mg/ml). Cell culture-grade dishes were from Greiner (Greiner 15 cm 639160) and NUNC (all other formats, e.g. 10 cm dish Nunclon A Surface, cat no. 150350; 6-well Nunclon A Surface, cat no. 140675). Cells were trypsinized and replated every 2nd day and frozen in FCS:ESCM:DMSO=4.5:4.5:1. Cells were tested for mycoplasma every second week. Etoposide treatment: Medium was supplemented every day with 3.3 nM etoposide, an LD₃₀ dose for 8 day treatment (Sigma E2600000), 1000× etoposide stocks dissolved in PBS-10% EtOH were used. For doxycycline treatment, medium was supplemented every day with 1 μg/ml (Sigma). Cells are tested for mycoplasma contamination every second week.

### Example 4: Viral Vectors and ES Cell Infection

For retroviral library generation, barcoded CRISPR library virus carrying a neomycin resistance cassette was packaged in PlatinumE cells (Cell Biolabs) according to manufacturers recommendations. Virus-containing supernatant was frozen at −80° C. 300 million ES cells were infected with a 1:10 dilution of virus containing supernatant for 24 h in the presence of 2 μg polybrene per ml (Sigma TR-1003). 24 h post infection, selection for infected cells started using G418 (Gibco) at 0.5 mg/ml. To estimate multiplicity of infection, 10,000 cells were plated on 15 cm dishes and selected using G418. For comparison, 1000 cells were plated but not exposed to G418 selection. On day 10, colonies were counted. After 24 h of Selection cells are split and 480 million cells are seeded on 60 15 cm dishes (Greiner 639160). After that cells are kept at a minimum cell number of 300 million cells during editing and screening.

### Example 5: iPS Cell Screen and Validations

Mouse embryonic fibroblasts containing Colla1::tetOP-OKSM, Oct4-GFP and Rosa26 M2rtTA alleles or Oct4-GFP alone were harvested from E13.5 embryos (Stadtfeld et al. Nature Methods 7, 53-55 (2010).). iPS cells were derived in DMEM supplemented with 15% FBS, 100 U/ml penicillin, 100 μg/ml streptomycin, sodium pyruvate (1 mM), 1-glutamine (4 mM), 1,000 U/ml LIF, 0.1 mM beta-mercapto ethanol, and 50 μg/ml ascorbic acid at 37° C. and 5% CO₂ as well as 4.5% O₂. MEFs were infected with a lentiviral vector delivering Cas9, selected for blasticidin resistance for 3 days, and subsequently infected with the sgRNA library. MEFs were treated with 0.5 mg/ml G418 for 3 days and 0.25 mg/ml G418 for an additional 3 days. For iPS induction, MEFs were plated at densities of 500,000 cells per 15 cm dish, and induced with doxycycline (1 μg/ml) for 7 days. After passaging for an additional 4 days in doxycycline-free media, Oct4-GFP-expressing cells were sorted from each replicate using a FACSAriaIII (BD Bioscience). All validation experiments were performed in 6 well dishes in triplicate, starting from 20 000 MEFs (Dox induction) or 40 000 cells (OKSM infection). Primary reprogramming was performed by infection with a lentiviral vector carrying OKSM factors as well as puromycin resistance and selected for puromycin resistance for 3 days. Oct-4 expression was quantified using a FACS BD LSR Fortessa (BD Biosciences), data were analyzed using FlowJo. Cells are tested for mycoplasma contamination every second week.

### Example 6: Etoposide Hypersensitivity Validations

For hit validation we used mouse embryonic stem cells expressing Cas9 under the control of doxycycline (T3G-Cas9-IRES-mcherry PGK-GFP-rtTA). We generated 4 knockout clones for the genes Lig4, Zfp451, Slc25a4, Adcy3, Rad9a, Erbb4 and 2 knockout clones for the genes Trim71 and Rac1 (sgRNAs, and PCR primers for genotyping) and tested every clone in triplicate. We pretreated cells for 7 days with 5 nM etoposide (Sigma E2600000) and then measured the drop of cell viability (treated/control) within a 3 day selection with 10 nM etoposide. Viability was measured with Alamar Blue staining (DAL1100 Thermo Fisher) according to manufacturer's recommendations.

### Example 7: DNA Harvest and NGS Sample Preparation

Readout of pooled CRISPR screens relies on precise PCR amplification of the integrated sgRNA cassette. However, in a genomic DNA prep the sgRNA cassette only makes up about 0.1 ppm of the total DNA. We improved this ratio by 3-4 orders of magnitude by flanking our sgRNA cassette with Pac-I sites (FIG. 4) and performing a size selective precipitation with digested genomic DNA. In detail: After 8 days of selection we lysed 170 million cells per condition using SDS-Lysis buffer (10 mM Tris pH8, 1% SDS, 10 mM EDTA, 100 mM NaCl)+1 mg/ml Proteinase K (Sigma P4032)+RNaseA (Qiagen). Genomic DNA was purified by phenol extraction, precipitated with isopropanol. Samples were digested with PacI (NEB R0547L). Size selective precipitation was carried out with using Speed Beads (GE45152105050250 Sigma Aldrich) according to manufacturer's recommendations. Each Sample was PCR amplified in 200 individual 50 μl PCR reactions using primers (AATGATACGGCGACCACC-GAGATCTACAC-NNNNNN-CGAGGGCCTATTTCCCATGATTCCTTC, SEQ ID NO: 2) where 6 bp are specific experimental indices used for demultiplexing samples after NGS sequencing and (CAAGCAGAAGACGGCATACGA-GATACCGTTGATGAGTAG, SEQ ID NO: 3). For qPCR analysis, we used GoTaq® qPCR Master mix (A6001 Promega) primers (AATGATACGGCGAC-CACCGAGATCTACACGAGTGGCGAGGGCCTATTTCCCATGATTCCTTC, SEQ ID NO: 4) and (CAAGCAGAAGACGGCATACGAGATACCGTTGATGAGTAG, SEQ ID NO: 5) for detection of a 579 bp amplicon on the 589 bp Pac-I Fragment with the CRISPR-UMI cassette and (GCCTTTAAGCCAATGCTAGCTG, SEQ ID NO: 6) and (GTAAATGGACAGAGGGTGTTTAACC, SEQ ID NO: 7) as a control with a 582 bp amplicon on a 7710 bp Pac-I fragment. PCR samples were purified on columns (Qiagen 27106) and pooled and size separated using agarose gel electrophoresis. The 600 bp band was excised and purified on a mini-elute column (QIAGEN 28204). This Sample was sequenced on an Illumina HiSeq2500 in a single-read 50 dual indexing sequencing run. We sequenced sgRNA sequences using 10× fold concentrated custom read primer (CGATTTCTT-GGCTTTATATATCTTGTGGAAAGGACGAAACACCG, SEQ ID NO: 8). For Analysis, it is necessary to obtain at least 10 bp for index 1 (barcode), and 6 bp for index 2 (experimental index).

Example 8: Data Analysis. For data analysis, we assigned sgRNA, unique molecular identifier sequences, and experimental indices to all reads using bowtie, samtools, fastx-toolkit and custom scripts. For Representation of guides in the library (FIG. 4f) library subpools were sequenced with individual experimental indices and the reads within each subpool were normalized to the median guide read of all subpools.

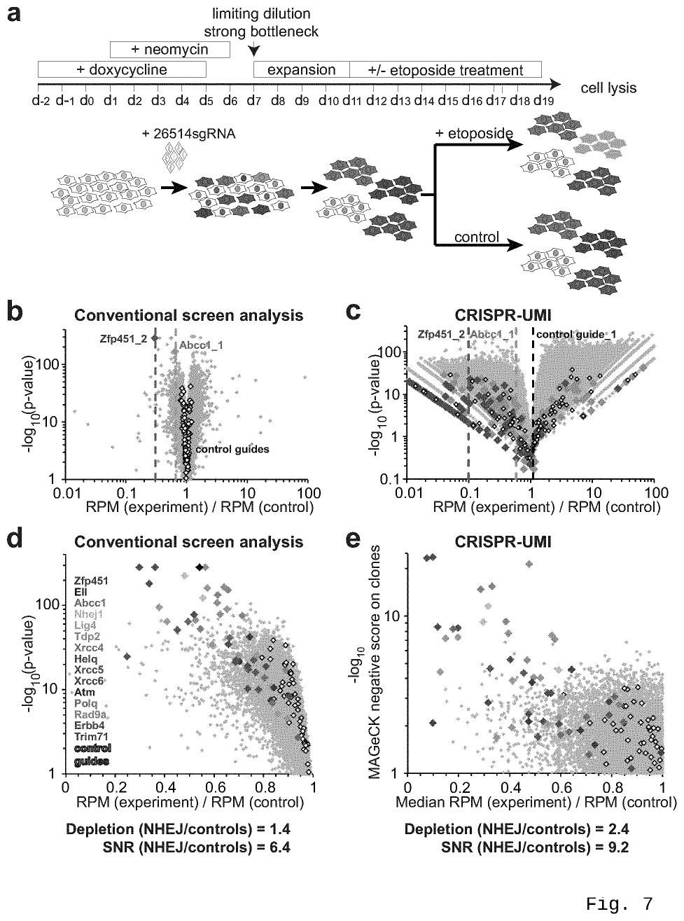

For conventional CRISPR data analysis (FIGS. 7b-d, 6d, 9a-c, 10b): We calculated depletion or enrichment of guides or clones using equation (1) and we added a pseudocount of 0.5 if there were no reads for a guide. P-values were calculated using cumulative binominal distribution functions (scipy.stats 0.19.0) using equation (2). We plotted depletion (x-axis) against p_{cdf} (y-axis) in a volcano plot.

$$d = \frac{{RPM}_{treated}}{{RPM}_{control}} \qquad ( 1 )$$

d . . . depletion of guide or clone

RPM . . . reads per million of that guide or clone

$${p}_{cdf} = {\sum }_{i = 0}^{x} ( \begin{matrix}n \\ x\end{matrix} ) {{p}^{x} ( 1 - p )}^{n - x} \qquad ( 2 )$$

p_{cdf} . . . p value cumulative binominal distribution function

x . . . reads of guide or clone in experiment

n . . . total reads in experiment

p . . . probability (reads of guide or clone in ctrl/total reads control)

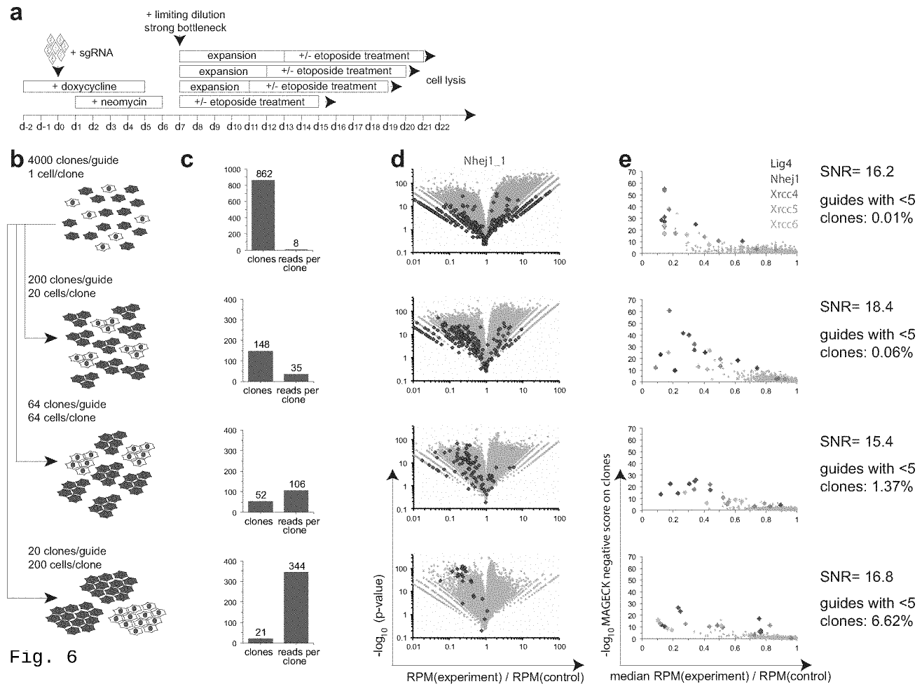

For clonal CRISPR-UMI analysis (FIG. 7e and FIG. 6e) we generated a “volcano-like” plot. We considered clones which contain at least 3 total reads (in treated and control combined) and guides with at least 5 clones present. To account for random barcode sequencing errors within experiments, we used adjacency-based barcode collapsing given an edit-distance of 1- and 3-fold read count difference using custom scripts. To combine data on clone level to data on guide level we plotted the median of depletion of clones against the MAGeCK neg score, a score for depletion for a sgRNA computed by using MAGeCK 0.5.5 (Li et al. Genome Biol. 15, 554 (2014)). Instead of using MAGeCK to calculate depletion-scores of a gene based on n guides, we used it to calculate depletion-scores of a guide based on n clones. Median depletion (x-axis) plotted against MAGeCK neg scores (y-axis) gives a “volcano-like” plot.

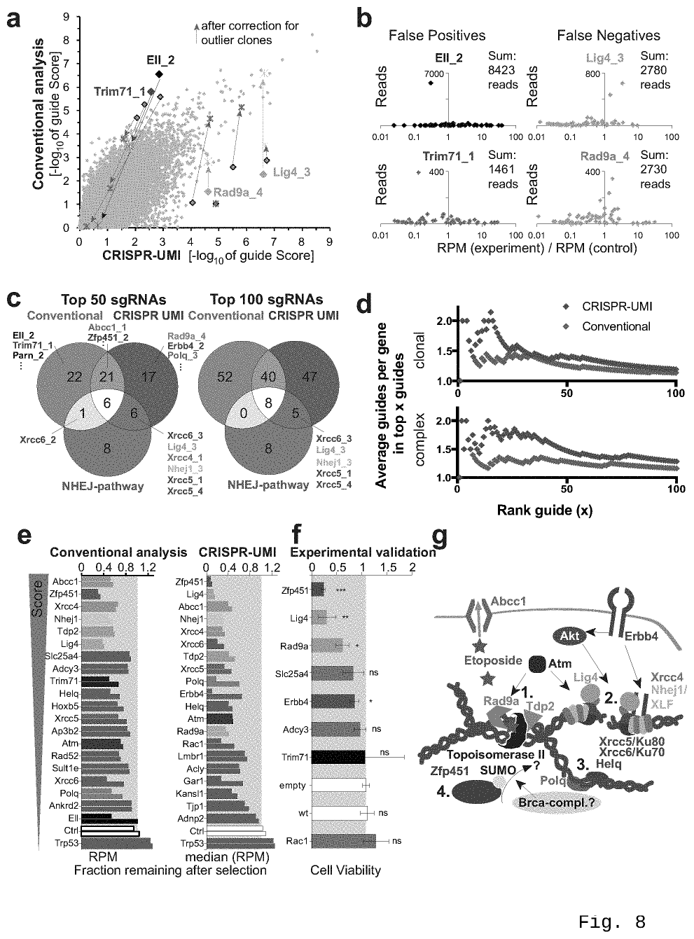

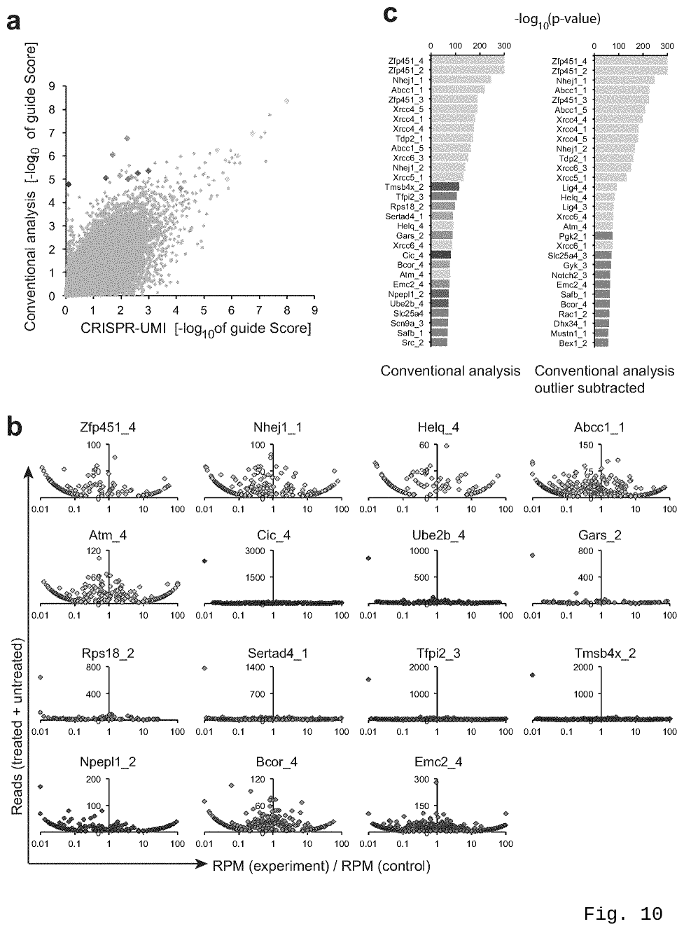

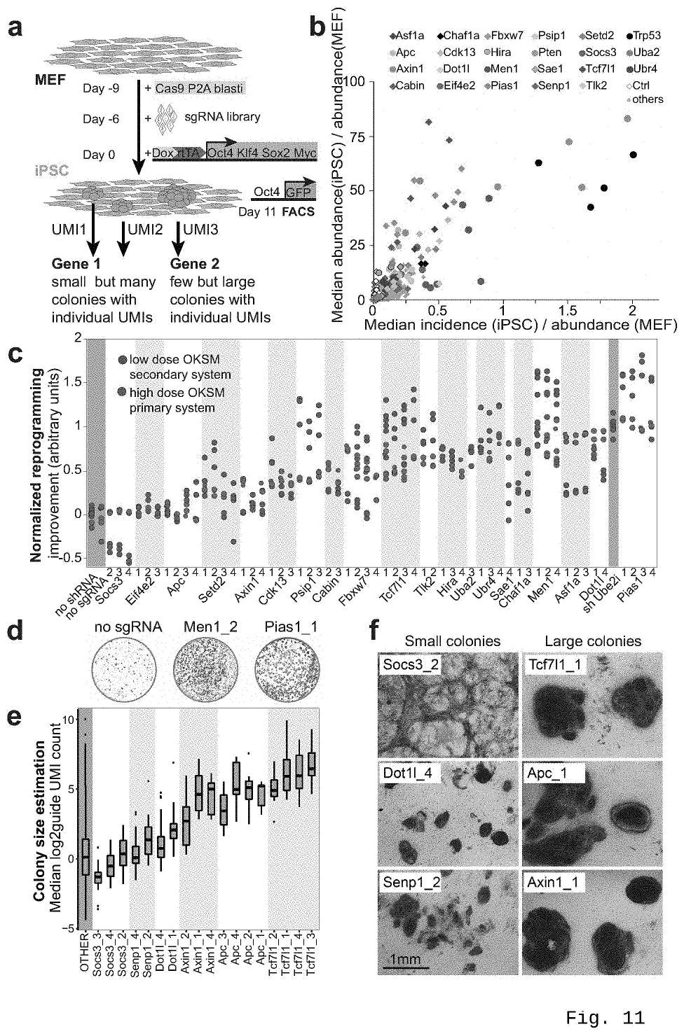

For performance comparison of conventional analysis vs CRISPR-UMI analysis on guide level (FIG. 8a and FIG. 10a) we ranked guides by both depletion and p-values for conventional analysis and by median depletion and MAGeCK neg scores for CRISPR-UMI analysis and then generated a combined Guide score using equation (3).

$$GS = \frac{{rank}_{depletion}}{{N}_{total guides}} \times \frac{{rank}_{p \text{-} value}}{{N}_{total guides}} \qquad ( 3 )$$

GS . . . Guide score−combined score, evaluation of guides

rank_{depletion} . . . rank of guide by depletion (Conventional Analysis)
rank of guide by median depletion (CRISPR-UMI Analysis)

rankₚ₋ᵥₐₗᵤₑ . . . rank of guide by p-value (Conventional Analysis)
rank of guide by mageck neg score (CRISPR-UMI Analysis)

Nₜₒₜₐₗ . . . guides total guides in analysis

For gene ranking (FIG. 3e) we used MAGeCK for conventional analysis and combined guide scores (GS) for CRISPR-UMI analysis using Fisher's method of p-value combination. For the top 5 genes with lowest p-values we could not apply Fisher's method due to numerical restrictions and sorted those 5 genes by combining p values using equation (4).

$${p}_{Gene} = \sqrt[n]{{\prod }_{i = 1}^{n} {GS}_{i}} \qquad ( 4 )$$

P_{Gene} . . . Score for Gene

GSᵢ . . . Guide score of guide i.

n . . . number of guides for that Gene

To calculate signal to noise ratios for a screen (FIGS. 3d and 3e and Supplementary FIG. 3e) we defined the signal of a guide as the distance from the origin of a volcano plot. This considers separation from noise in both depletion (x-axis) and significance (y-axis). In the volcano plot the x-axis is the ratio of guide reads in treated over control (for CRISPR-UMI the median of many clones) and the y-axis is the negative logarithm of the p-value as determined by binominal distribution in conventional analysis or the negative score as determined by MAGeCK for multiple clones per guide in CRISPR-UMI Analysis. Distance on the x axis was normalized to the guide with strongest depletion in the experiment. Distance on the y-axis was normalized to the guide with best significance score. Signal is the diagonal distance of the x-normalized and y-normalized distance from the origin calculated using equation (5). Signal to noise ratios are calculated using equation (6).

$${S}_{i} = \sqrt{{( \frac{1 - {d}_{i}}{1 - {d}_{min}} )}^{2} + {( \frac{{log}_{10} ( {p}_{i} )}{{log}_{10} ( {p}_{min} )} )}^{2}} \qquad ( 5 )$$

Sᵢ . . . signal of guide i.

dᵢ . . . depletion of guide i (Conventional)
median depletion of clones for guide i (CRISPR-UMI)

dₘᵢₙ . . . depletion of strongest depleting guide in the comparison (Conventional),
lowest median depletion of all guides in the comparison (CRISPR-UMI)

pᵢ . . . p-value for depletion for guide i (Conventional),
mageck neg score for guide i (CRISPR-UMI)

pₘᵢₙ . . . lowest p-value of all guides in the comparison (Conventional),
lowest mageck neg score for all guides in the comparison (CRISPR-UMI)

$$SNR = \frac{{\overline{s}}_{NHEJ}}{{σ}_{CTRL}} \qquad ( 6 )$$

SNR . . . signal to noise ratio.

s̅_{NMEI} . . . average signal of all guides of NHEJ pathway (Lig4, Nhej1, Xrcc4-6)

σ_{ctrl} . . . Standard deviation of signal from all control guides

We evaluated CRISPR-UMI vs conventional screen using a value termed Depletion (NHEJ/control) (FIGS. 7d and 7e). That's a ratio of the geometric mean of fold change of all guides against NHEJ-pathway genes (Lig4, Nhej1, Xrcc4-6) and the geometric mean of fold change of non-targeting control guides.

To assess the efficiency of different screening and analysis methods (FIG. 8d), we evaluated the ranking of guides by guide scores GS calculated using equation 3. We determine the average number of guides present per gene among the top guides (y axis) while expanding the list of top guides from 1 to 100 (x-axis). E.g. If the among the top 30 guides (x-axis) there are guides against 15 different genes there are in average 2 (y-axis) guides per gene.

For incidence vs abundance analysis of a positive selection screen of reprogramming MEFs to iPSC (FIG. 11b) we determined read counts for all individual UMIs. Read counts for all UMIs belonging to the same guide were added up for a measure of abundance for each guide. The number of independent UMIs per guide passing the threshold criteria for a iPSC colony (at least 10 reads) is a measure for incidence. Enrichment for guides in either abundance or incidence is normalized to abundance of the guides in the starting MEF population. Each data point shown is the median of 4 biological replicas.

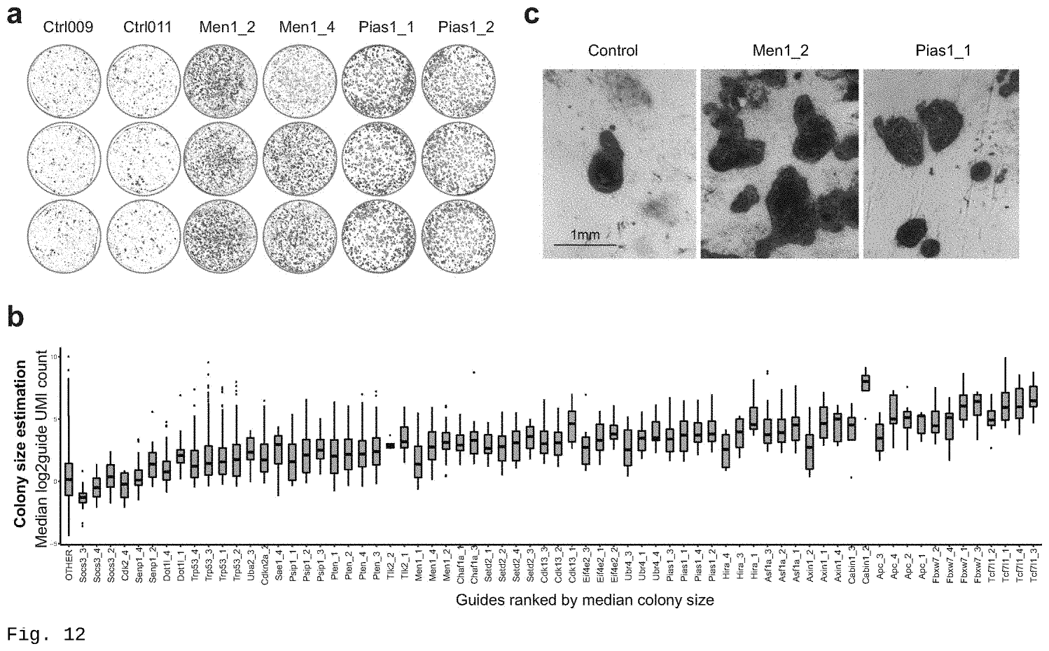

To estimate colony size of iPSC (FIG. 11e and FIG. 12) read counts for all UMIs were determined, and scaled by library size across 8 treated and 4 untreated replicates. UMI barcodes with a hamming distance of 1 were collapsed to account for UMI sequencing errors using UMI-tools (Smith et al. Genome Res. 27, 491-499 (2017)). UMIs with low counts were filtered by retaining UMIs with 5 or more read counts. Starting with the highest represented guide-UMI combination we included all UMIs up to a cumulative percentage of 90% per guide. Normalized UMI counts were divided by the median of the UMI counts for each sample, the values from the 8 treated replicates were pooled, log-transformed and used to visualize the distribution of the abundance estimates relative to the experimental median for each guide.

### Example 9: Statistical Information

All error bars are standard deviation or standard error of the mean as indicated in FIG. 30 legends. Number of experiments n is given for every experiment. Statistical information for conventional and CRISPR-UMI screen-analysis is described in data analysis section.

### Example 10: A Framework for Single Cell-Based CRISPR Screening

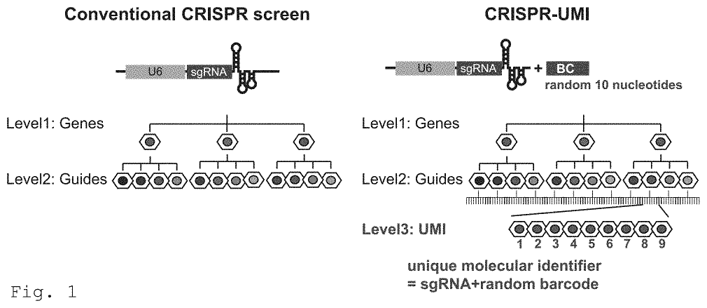

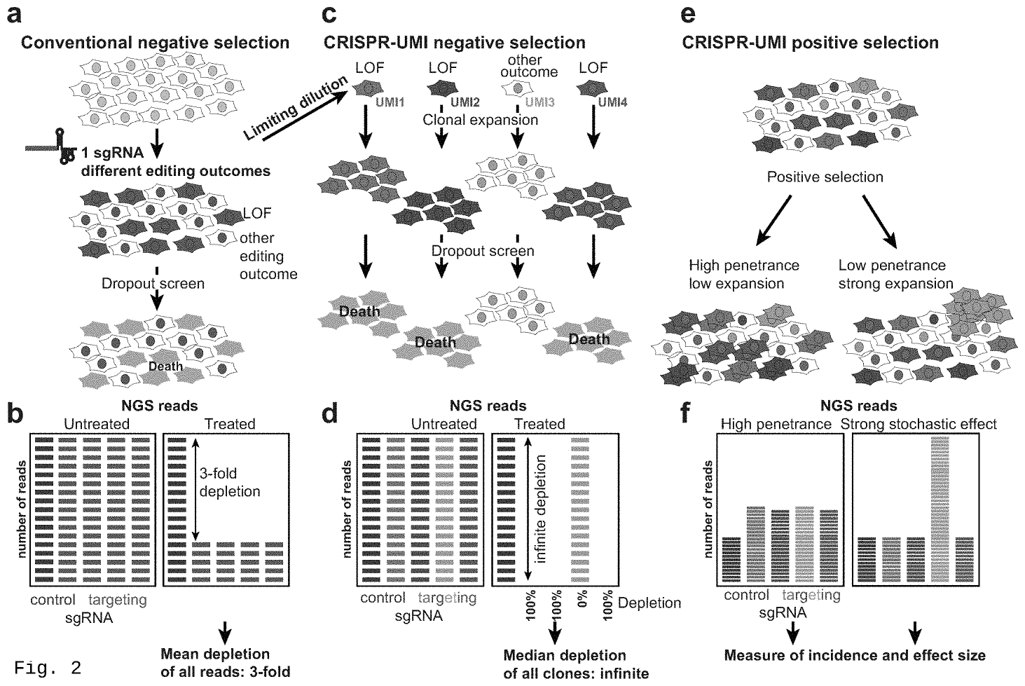

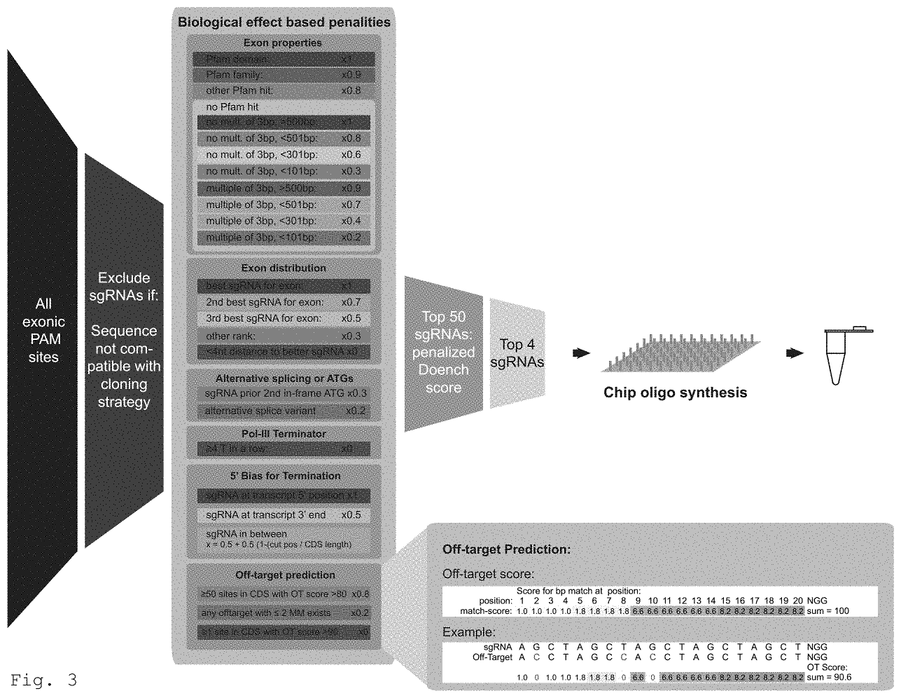

Conceptually, the depletion limit in CRISPR screens exists only on the population level. On a single cell level, cells either harbor homozygous LOF alleles, whereas others harbor any combination of alternative outcomes (Shalem et al. Nat. Rev, Genet. 16, 299-311 (2015)). In other words, the genetics at single cell level can be considered binary, while the genetics at population level is graded depending on guide efficiency. To track individual cells, we added a random barcode, that together with the sgRNA generates a “unique molecular identifier” (UMI) (Kivioja, T. et al. Nature Methods 9, 72-74 (2011)), thus increasing the depth for CRISPR screening (FIG. 1). We evaluate independent biological replicates within a pooled CRISPR screen by following single cell-derived genetically marked clones after limiting dilution (FIG. 2c). This approach discerns clones with homozygous LOF alleles that drop out completely from clones with non-LOF alleles that survive selection (FIG. 2d), and thus overcomes the expected depletion limit of a conventional population based screen which does not discern LOF from non-LOF alleles (FIG. 2b). Importantly, response of LOF clones to selection is a true reflection of biological effect and no longer be overlaid with editing efficiency. This binary mode of scoring can be leveraged if i) cells infected with a sgRNA can be distinguished from one another by use of a random barcode/UMI, and ii) the cell population is carried through a strong bottleneck so that a maximum of 1 cell and thereby 1 editing outcome remains in the population for each independent infection event (UMI), termed clone. We call this method CRISPR-UMI to highlight the presence of unique molecular identifiers allowing for tracking of each cellular event.

Beyond providing improved resolution for LOF screens, CRISPR-UMI also enables analysis of population behavior. For instance, the enrichment of an sgRNA sifter positive selection can arise from either massive expansion of a few, single cells, or milder expansion of all cells within the population carrying a specific sgRNA. However, the frequency of such stochastic events cannot be deduced from conventional population based CRISPR screen analysis. In contrast, CRISPR-UMI allows for the assessment of population behavior and thus for quantification of effect size and probability. Therefore, it generates increased information content from screening readouts (FIG. 2e, f).

### Example 12: Generation of a Complex CRISPR Library Using Random Barcodes

To ensure optimal sgRNA efficiency, minimal off targeting, as well as a likely biological effect on target for our CRISPR-UMI library, we predicted Doench-scores (Doench, J. G. et al. Nature Biotechnology 32, 1262-1267 (2014)) for ail possible sgRNA within the genome and subsequently factored in additional parameters such as off-target predictions, position within the on-target transcripts, exon structure, and protein coding domains (FIG. 3).

CRISPR-UMI requires the generation of a high complexity library for clonal tracking of individual cells. We generated a retroviral vector and introduced a stretch of 10 random nucleotides by parallel cloning (1*10⁶ bacterial colonies, reaching the theoretical maximum complexity of a 10 nt UMI 4¹⁰=1.048*10⁶) and confirmed presence of random barcodes by NGS. Subsequently, several sub-poo Is of sgRNAs targeting a total of 6560 different, mouse genes including all nuclear genes with 4 sgRNAs/gene were cloned into the vector-barcode-library at a coverage of 954-8776 independent cloning events per sgRNA. We also included a set of 112 non-targeting control sgRNAs to the library (Wang et al. Science 343, 80-84 (2014)) (FIG. 4a, b, FIG. 5, and Methods in Examples 1-10). Thus, each sgRNA is combined with a different barcode in each ligation event. The combination of sgRNA and barcode together represent the unique molecular identifiers (UMI). The overall library complexity reached 83.5 million, which exceeds the number of clones assayed within a screen and thus allowed for tracking of individual cells in subsequent genetic screens. To account, for the large quantities of genomic material that need to be processed subsequent to genetic screens, the vector design further allows for enrichment of genomic DNA containing sgRNA integrations prior to PCR amplification by PacI endonuclease digest and subsequent size selection (FIG. 4c, see Methods and step-by-step protocol). This step enriches the PCR-templates 10³ to 10⁴-fold (FIG. 4d) and thereby minimizes the required number of PCR reactions and cycles, and thus PCR amplification biases. By integration of specific sequence stretches, the vector design further allows the direct use of standard illumina primers (FIG. 4e).

We applied our sequencing strategy on plasmid DNA of our libraries to analyze the representation of sgRNAs and UMIs. The number of distinct sequenced UMIs per sgRNA correlated with the estimation of cloning depth based on number of bacterial colonies obtained. The relative difference in abundance of guides in the library at the 10th and 90th percentile is 4-fold (FIG. 4f), which compares well to other published sgRNA libraries (Koike-Yusa et al. Nature Biotechnology 32, 267-273 (2013), Wang et al. Science 343, 80-84 (2014) and Hart, T. et al. Cell 163, 1515-1526 (2015)). Taken together, we generated libraries targeting mostly protein coding domains of nuclear proteins with even distribution and highly complex UMIs that allow for single cell based CRISPR screens.

### Example 13: Single Cell Lineage Tracing Improves Signal-to-Noise Ratio in Dropout CRISPR Screening

To test CRISPR-UMI, we exposed cells to the chemotherapeutic drug etoposide, which generates DNA double strand breaks via inhibition of topoisomerase II (Burden, D. A. et al. Journal of Biological Chemistry 271, 29238-29244 (1996)). Cells that are defective in double strand break repair pathways, such as non-homologous end joining (NHEJ), are expected to be sensitive to this treatment (Srivastava, M. et al. Cell 151, 1474-1487 (2012), Kurosawa, A. et al. PLoS ONE 8, e72253 (2013), Fattah, F. J. et al. DNA Repair (Amst.) 15, 39-53 (2014) and Jackson, S. P. & Bartek, J. Nature 461, 1071-1078 (2009)).

To optimize the conditions for single cell derived clonal CRISPR screening, we performed a pilot screen on a subset of 365 genes (1437 guides) associated with DNA damage response. We infected mouse embryonic stem, cells (mESCs) harboring a doxycycline (Dox)-inducible Cas9 cassette with retroviral vectors delivering our sgRNA library and selected for G418 resistant clones. Clone size and number can be modified by varying the limiting dilutions as well as the time of clonal expansion of infected cells (FIG. 6a, b). Importantly, the number of NGS reads required for the CRISPR-UMI screen does not exceed the number needed for conventional analysis. Given this limited sequencing space for a full-scale experiment, we tested a matrix of 4000 reads/guide in 4 conditions and aimed for i) 20 clones of 200 reads, ii) 64 clones of 64 reads, iii) 200 clones of 20 reads and iv) no limiting dilution or expansion resulting in 4000 mostly independently infected cells (FIG. 6c, see Methods in Examples 1-10). In each setup, we evaluated the performance of positive control guides targeting core members of the NHEJ complex. We combine the median depletion of individual clones (FIG. 6d) per guide, as well as a p-value for depletion using MAGeCK 0.5.5 (Li, W. et al. Genome Biol. 15, 554 (2014)) that ranks individual clones for one guide relative to the full dataset to evaluate significance of bias towards depletion (FIG. 6e). Thus, we combined many individual clones carrying the same guide using MAGeCK similarly to combining independent guides targeting the same gene. In doing so, we reached one level deeper into the screen, namely down to clonal analysis. We identified all core members of the NHEJ complex, with multiple guides scoring highly significant, validating quality of the guide prediction and library. We next compared the different conditions of clone sizes. To identify the optimal compromise between number of clones and p-value per clone, we quantified performance as signal-to-noise ratio (SNR) of NHEJ pathway members relative to control sgRNAs by calculating the distance of each sgRNAs to the origin of the volcano plot (FIG. 6e). SNR reached similar values of 15-18 in all conditions, but was best at 148 clones of 35 reads. Importantly, guide representation translates into a variable number of clones per guide, while clone size is not affected by sgRNA abundance. We estimated based on the sgRNA distribution in our library (FIG. 4f), that 148 clones of 35 reads would also result in the lowest number of guides represented in too few clones for analysis (0.06% versus 1.4% or 6.6%, FIG. 6e). We therefore concluded that the ideal parameters for single cell lineage tracing CRISPR screening in our setting are roughly 150 clones with on average 30-40 reads, however this might vary depending on application.

Using this optimized clone number and size, we next tested CRISPR-UMI in an etoposide screen with the full library of 26514 guides targeting 6560 genes, containing all predicted nuclear genes in the mouse genome, as well as orthologues of drugged human genes (FIG. 7a). Using the same mESC line carrying Dox-inducible Cas9, we compared performance at the guide level as a measure of sensitivity and specificity in CRISPR-based negative selection screens. Depletion levels and p-values of depletion for pools versus single cell derived clones were evaluated and we observed clonal variance as well as increased levels of depletion at the single cell level (FIG. 7b, c).

To determine if CRISPR-UMI outperforms conventional analysis, we combined all clones per guide and plotted median depletion for each guide versus a p-value computed using the robust ranking algorithm of MAGeCK0.5.5 (Li, W. et al. Genome Biol. 15, 554 (2014)) (FIG. 7e, compare to 7d). Median depletion of multiple individual clones mostly representing LOF clones is expected to give a better measure for biological effect (see also FIG. 2e). Indeed, CRISPR-UMI resulted in a better separation of signal from noise (SNRCRISPR-UMI=9.2 versus SNRCRISPR=6.4 for NHEJ/control) and stronger depletion (Depl.CRISPR-UMI=2.4 versus Depl.CRISPR=1.4 for NHEJ/control) compared to conventional analysis (FIG. 7d, e).

To directly compare performance of conventional versus CRISPR-UMI analysis, we ranked guides within each dataset (see Methods). We observed a high degree of correlation between conventional and single-cell based screen analysis (Pearson correlation: 0.751), but we also observed guides that were uncovered only with conventional or clonal CRISPR analysis (FIG. 8a).

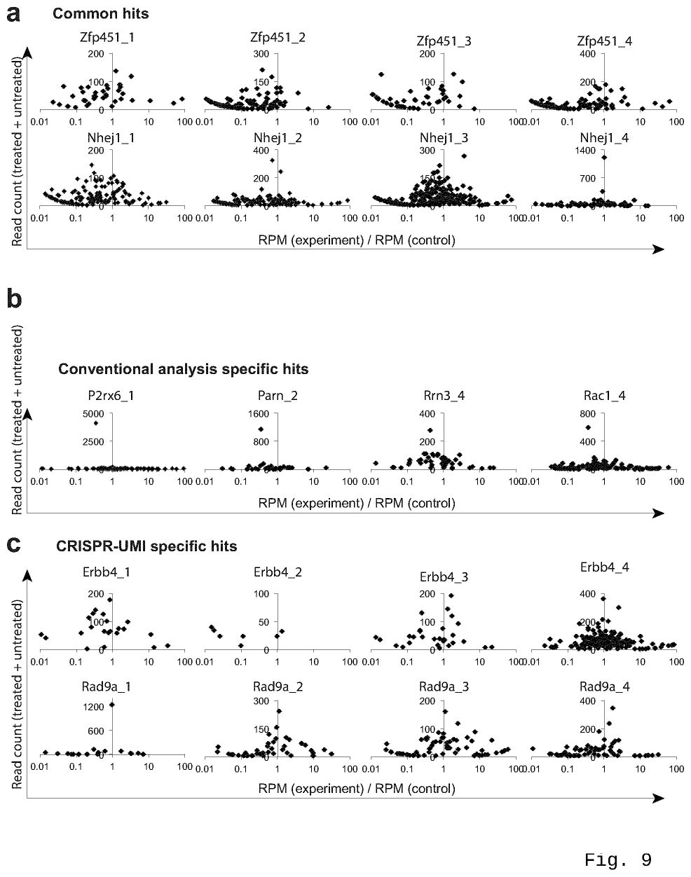

Intriguingly, we found that discrepancies are due to strong outlier clones with vastly overrepresented read numbers dominating the total read space in all discrepant cases (FIG. 8b and FIG. 9). These strong outliers interfere substantially with conventional analysis, which assumes that all cells within the experiment are equally represented. For guide 1 against. Trim71 and guide 2 targeting Ell, that only scored in conventional analysis, outlier clones snowed reduced read count in the treated sample compared to the control. These outliers reduced the total reads for these guides, masking the fact that all other clones of these guides showed no obvious tendency for depletion (FIG. 8b). In contrast, guides that were exclusively identified by CRISPR-UMI, such as Lig4 guide 3 and Rad9a guide 4, were associated with outlier clones having increased read counts in treated cells compared to the control, resulting in increased total reads for this guide, masking the depletion of most of the clones of these guides. We thus hypothesized that these outlier clones cause the discrepant results obtained between conventional and clone-based analysis.

To test this hypothesis, we removed the 2 clones with most reads from the data of each guide and reanalyzed all data using both methods. This resulted in a realignment of both analysis pipelines (see asterisks in 8a, curated Pearson correlation: 0.764). Upon removal of the outliers, the guides that were previously only identified by CRISPR-UMI were now also uncovered by conventional analysis, whereas the guides that were previously identified uniquely by conventional analysis dropped to a lower position in the conventional MAGeCK ranking of genes (Trim71: 9 to 214; Ell: 20 to 4416) upon outlier removal. Thus, the guides uniquely identified by population based methods were putative false positives, and those missed by conventional methods were putative false negatives. We conclude that in this screening regimen, outlier clones, which usually remain undetected, confounded conventional screen analysis but not CRISPR-UMI. We considered that the limiting dilution step in our protocol might underlie the observation of outlier clones. To investigate this possibility, we performed the same etoposide dropout screen without limiting dilution. A comparison of both scoring algorithms again revealed several guide RNAs that were differentially called by conventional analysis versus CRISPR-UMI. Once more, these discrepancies were due to outlier clones and highlighted shortcomings in conventional analysis (FIG. 10). Taken together, outlier clones are not introduced by limiting dilution. CRISPR-UMI analysis of pooled screens is thus superior to conventional analysis as it avoids putatively false positive or negative calls arising from clonal variation and outliers.

To compare the performance of both scoring methods directly, we asked how many of the positive control sgRNAs designed to target the NHEJ complex (Lig4, Xrcc4-6, Nhej1) score amongst the top 50 or 100 guides (FIG. 8c). Whereas conventional analysis only calls 7 and 8 out of 21 sgRNAs respectively, CRISPR-UMI scores 12 and 13 sgRNAs within the top 50 or top 100. Next, we determined the reproducibility on sgRNA level of all genes identified in the screen. We plotted the average number of sgRNAs present per gene (for all genes hit by the respective group of guides) as a function of increasing numbers of sgRNAs according to rank (FIG. 8d, e.g. if by the top 30 sg RNAs, 15 genes are hit, the value is 2; a value of 1 would be expected for a random dataset). For both full library screens (with and without clonal dilution-expansion) CRISPR-UMI clearly outperformed conventional analysis across the entire hit list and showed higher reproducibility between sgRNAs.

To evaluate the results of each method of analysis on the gene level, we combined guides using MAGeCK for conventional analysis and Fisher's method for CRISPR-UMI (see Methods) and ranked them according to score within each method (FIG. 8e), Both methods scored multiple expected hits in DNA repair pathways, which we color-coded according to function (FIG. 7g) (Jackson, S. P. & Bartek, J. Nature 461, 1071-1078 (2009), Black et al. Genes (Basel) 7, 67 (2016) and Takata et al. Nat Commun 4, 2338 (2013)). Furthermore, both methods identified a specific proton pump, Abcc1, as well as a novel SUMO E3 ligase, Zfp451. CRISPR-UMI outperformed conventional analysis methods not only on guide level but also on gene level, showing stronger level of depletion and better ranking of nits. To test if the genes identified in the negative selection screen indeed validate experimentally, we derived several independent KO cell lines for common hits (Lig4 and Zfp451) as well as conventional analysis specific (Slc25a4, Adcy3, Trim71) and CRISPR-UMI specific hits (Rad9a, Erbb4, Rac1) and quantified dropout in response to etoposide relative to control (FIG. 4f). Both common nits showed strong drop out, also Rad9a and Erbb4 depleted significantly, while the conventional analysis specific hits did not validate. Of note, effect size in screen readout and validation also correlated well. Reassuringly, genes identified by CRISPR-UMI, namely Rad9a and Erbb4 (FIG. 8e and FIG. 9c), were both previously implicated in DSB repair or decatenation of DNA in response to TopoII inhibition (Gilmore-Hebert et al. Mol. Cancer Res. 8, 1388-1398 (2010), Icli et al. Biochem. Biophys. Res. Commun. 418, 116-121 (2012), Mukherjee et al. Seminars in Radiation Oncology 20, 250-257 (2010), Greer et al. J. Biol. Chem, 285, 15653-15661 (2010), He et al. Nucleic Acids Res. 39, 4719-4727 (2011) and Smilenov et al. Cancer Res. 65, 933-938 (2005)).

Taken together, we identified multiple known SLS well as novel proteins involved in DNA repair upon etoposide-mediated topoisomerase II inhibition. Moreover, we snow, that the quality of screening results obtained by CRISPR-UMI exceeds the one generated conventionally both in robust identification and quantification of phenotypes.

### Example 14: Positive Selection Screen to Elucidate Roadblocks of Reprogramming

Next, we chose a robust iPS cell induction protocol (Stadtfeld et al. Nature Methods 7, 53-55 (2010)) to test CRISPR-UMI during a stochastic single cell positive selection paradigm. The clonal dilution step was omitted from this approach to keep barcode complexity as high as possible, as the stochasticity of IPS induction replaced the limiting dilution and generates the clones for CRISPR-UMI analysis (FIG. 11a, compare to 2e, f). We collected mouse embryonic fibroblasts (MEFs) that contain a Dox-inducible Oct4-Klf4-Sox2-Myc (OKSM) cassette as well as an endogenous Oct4-GFP reporter, which can serve as proxy of successful reprogramming. We infected these MEFs with a lentiviral construct delivering Cas9 and subsequently our sgRNA barcode library (FIG. 11a). Six days post infection (day 0), we induced OKSM by Dox treatment for 7 days. Cells were then passaged and cultured without Dox assay IPS fate independent of exogenous OKSM, followed by FACS-purification of Oct4-GFP positive cells on day 11 to isolate IPS cells. Subsequently, we isolated genomic DMA from iPS cells (“treated”) and MEFs (“untreated”) and determined sgRNA abundance as well as the number of incidents of independent guide-barcode combinations (i.e. IPS colonies) (FIG. 11b). The incidence, reported by the number of independent barcodes, reflects probability of IPS colony formation, whereas sgRNA read abundance reports total amount of IPS cells. We identified many known roadblocks of reprogramming such as Trp53 (Marion et al. Nature 460, 1149-1153 (2009)), Pten (Liao et al. Mol. Ther. 21, 1242-1250 (2013)), Dot1l (Onder et al. Nature 483, 598-602 (2012)), Socs3 (Buckley et al. Cell Stem Cell 11, 783-798 (2012)), Sae1, Uba2 and Chaf1a (Cheloufi et al. Nature 528, 218-224 (2015)) (FIG. 11b). In the inventive aspects, such known reprogramming targets are preferably not used alone but in combination with another reprogramming target of the invention, such as Menin. Of note, guides against Senp1, Socs3, and Dot1l primarily scored on the incidents axis, and in Trp53 almost 100% of UMIs gave rise to IPS cell colonies, presumably due to the expansion of the MEF population prior to reprogramming. This highlights the additional resolution gained from, this additional readout parameter.

We also identified several novel candidates for roadblocks of reprogramming and performed validation experiments for 20 genes (FIG. 11c). As reprogramming is strongly dependent on timing and dose of OKSM (Cheloufi et al. Nature 528, 218-224 (2015)), we used two approaches for validation: (1) low OKSM levels obtained the Dox-inducible OKSM MEFs, mimicking the original screen and (2) high OKSM levels obtained by lentiviral OKSM delivery. We included knockdown of Ube2i as positive control, which improved reprogramming >100-fold in this setting. As expected, almost all sgRNAs enhanced reprogramming efficiency in the screening approach (FIG. 11c, d and FIG. 12a). However, the effect size for each gene varied between both reprogramming systems, in agreement with prior findings, suggesting system-specific roadblocks of the IPS reprogramming process (Santos et al. Cell Stem Cell 15, 102-110 (2014) and Rais et al. Nature 502, 65-70 (2013)). Importantly, targeting of novel genes such as Pias1, an E3 SUMO-protein ligase, and Men1, encoding for the transcriptional cofactor Menin, markedly outperformed all tested and previously identified roadblocks in a primary reprogramming regimen. Taken together, our screening approach based on a combination of abundance of guide RNA as well as independent clones identified multiple known as well as novel roadblocks of reprogramming.

We next wanted to make further use of our barcodes to more deeply analyze data from the primary screen, and focused on those sgRNAs that most significantly enhanced reprogramming efficiency. We plotted median iPS colony size by quantifying the read count for each barcode-tagged individual colony (FIG. 11e, FIG. 12b), Of note, colony size reflects the reprogramming speed and/or growth kinetics of resultant iPS colonies and is different from the stochastic probability of establishment of colonies, both parameters together result in the increased abundance of sgRNA reads. Interestingly, we observed a distribution of read counts that was gene specific and relatively reproducible between sgRNAs. This result was immediately suggestive of biology and predicts that distinct colony sizes will be obtained with different guide RNAs. To test if read count distribution reflects true biological outcomes regarding the incidence of iPS colony formation versus the size of such colonies, we imaged colony appearance 10 days after Box induction for guides that were predicted to generate particularly large or small colonies. Indeed, the observed colony size perfectly correlated with expected size distribution from primary screen analysis (FIG. 11f and FIG. 12c). In summary, we show that single cell based CRISPR analysis in a regimen of stochastic positive selection can robustly identify nits, as well as predict the variation of probability over variation of event quantity. We snow that new reprogramming targets to iPS cells generation have been found, which were further validated in an iPS cell assay.

### Example 15: Genetic Modulation of Human Reprogramming Efficiency

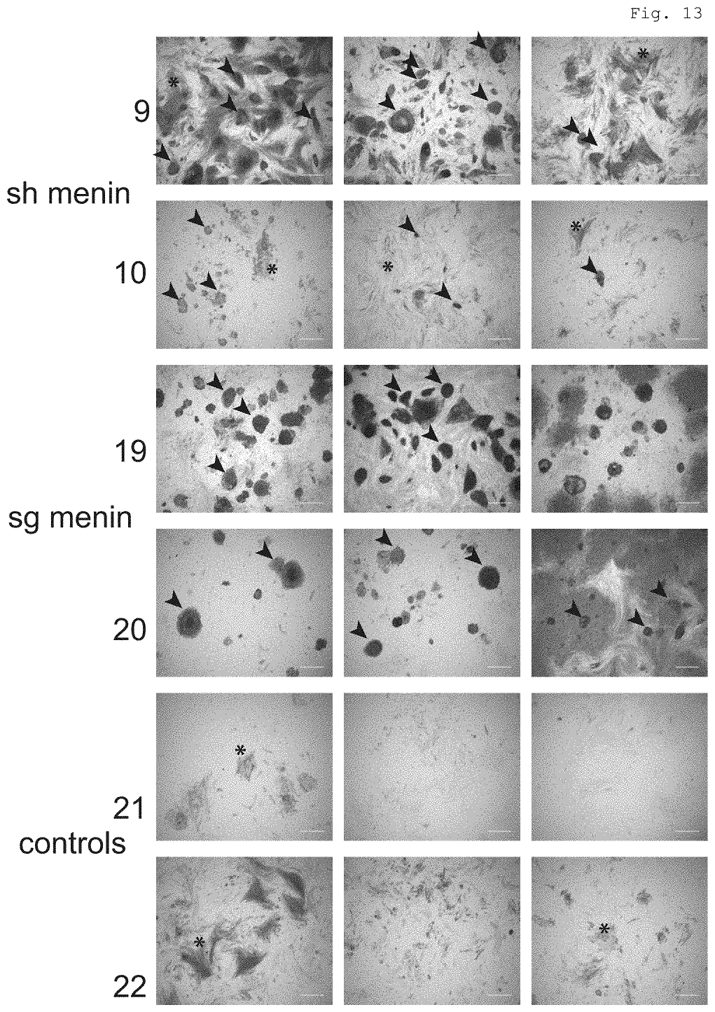

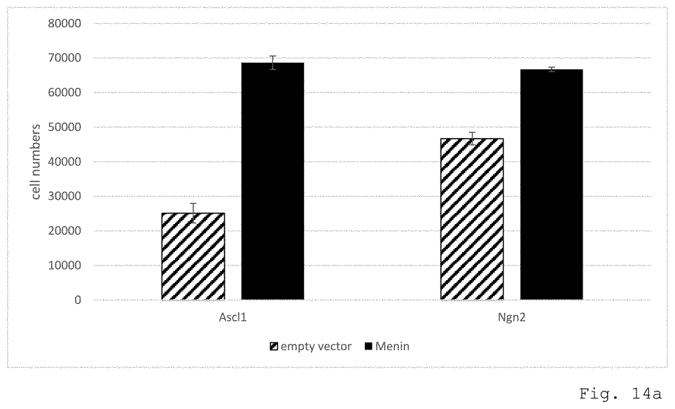

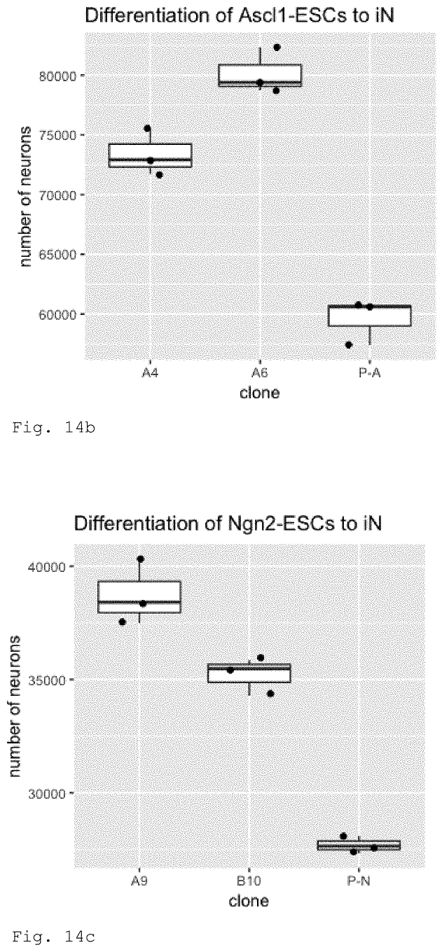

Primary human dermal fibroblasts were infected with lentiviral constructs carrying either knockdown shRNAs constructs, or sgRNAs plus Cas9 to genetically target gene loci. These constructs additionally carried a blasticidin selection cassette. Subsequently, fibroblasts were selected for successful infection and infected again 46 days after the initial infection with a lentiviral vector carrying an expression cassette for the 4 “Yamanaka” factors Oct4, Klf4, Sox2, and Myc (OKSM) coupled to a puromycin selection cassette. Puromycin selection was initiated the day after. Six days after OKSM infection, cells were trypsinized and transferred on a feeder cell layer consisting of CF-1 cells. Cells were maintained for another 2 weeks to evaluate reprogramming efficiency by alkaline phosphatase (AP) staining, which is shown in FIG. 13. AP stains iPS cell colonies dark blue (arrowheads), while fibroblasts do not stain or appear as fibroblastic stained cells (asterisk).

Reprogramming efficiency in empty shRNA or empty sgRNA control vectors was low as expected. While sgRNAs against CHAF1A, SAE1, and TJBE2I, did not enhance reprogramming efficiency, reprogramming was markedly improved upon knockdown of Menin mRNA or -more pronounced-editing of the MEN1 locus. In conclusion, Menin activity prevents efficient reprogramming of human dermal fibroblasts, inhibition of MENIN thus presents an efficient method of enhancing reprogramming in human samples.

### Example 16: Induced Differentiation of ESC to iN (Induced Neurons)

Expression of proneural factors Ascl1 and Ngn2 leads to direct conversion of embryonic stem cells (ESC) to neurons without intermediate states. In comparison to previous pure growth factors induced differentiation, this regime allows generation of neurons in a simpler (one step protocol compared to multi-step differentiation protocols with multiple medium conditions), faster (only 4-5 days for generating beta-III-tubulin (Tuj) positive cells compared to 7-14 days, e.g. in Gaspard et al., Nature Protocols 4(10), 2009: 1454-1463), near 100% purity and having more uniform neuronal subtype as end point differentiation. Furthermore, this method is more cost effective due to the reduced requirements in growth factors.

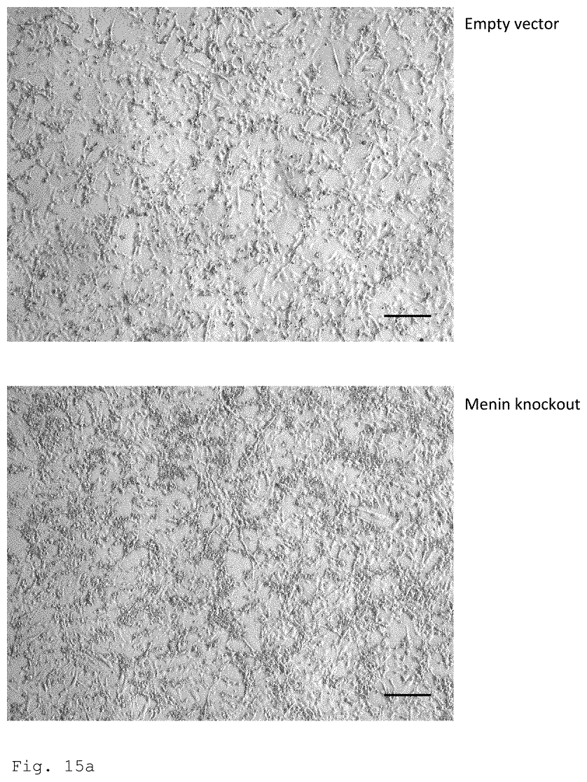

ESC carrying a doxycyclin inducible Ascl1 (Achaete-Scute Family BHLH Transcription Factor 1) or Ngn2 (Neurogenin 2) cassette (Ascl1-ESCs and Ngn2-ESCs) and constitutively active Cas9 were infected with retrovirus carrying guide against Menin to introduce menin knockout. ES cells were plated at clonal density and individual colonies were picked and genotyped to confirm homozygous Menin knockout. The corresponding clones were expanded and exposed to 7 days of doxycycline treatment. From, day four on, cells were treated with the drug AraC (Cytosine β-D-arabinofuranoside) to eliminate dividing cells and purify the neuron population. At day 7 of dox treatment, cells were analyzed using fluorescence activated cell sorting (FACS) for the expression of endogenously tagged pan-neuronal gene MAPT (Microtubule-Associated Protein Tau) with P2A-Venus reporter. Cell numbers were compared between menin knockout ESC and ESC without menin knockout. Data were acquired from three biological triplica. The first plot in FIG. 14 illustrates the mean number of iN derived from Ascl1 and Ngn2 cell lines with and without menin knockout. The boxplots of FIGS. 14 (b) and (c) show data from two clones with confirmed homozygous menin knockout and the corresponding parent cell line without menin knockout. The data shows that neuronal transdifferentiation using neuronal transcription factors Ascl1 or Ngn2 can be enhanced by Menin inhibition.

### Example 17: Transdifferentiation MEF to iN

Enforced expression of transdifferentiation inducing genes is currently the only method to convert MEFs into functional neurons besides the detour via reprogramming using e.g. Yamanaka-factors. Furthermore, transdifferentiation is devoid of teratoma formation, which can arise from incomplete neuronal differentiation from ESC. MEFs (mouse embryonic fibroblasts) carrying and inducible Ascl1 cassette (Ascl1-MEFs) were infected with viruses carrying Menin guides and Cas9 to introduce menin knockout. Cells were plated on coverslips covered with a layer of P53-knockdown immortalized primary glia obtained from P3 mouse pups. After 13 days of doxycycline treatment, cells were fixed, using 4% PFA and stained for the pan neuronal marker beta-III-tubulin (Tuj). Number of Tuj-positive neurons per defined area (1.64 μm²) on the coverslips was obtained from confocal images and manual cell counting tool of the Fiji software.

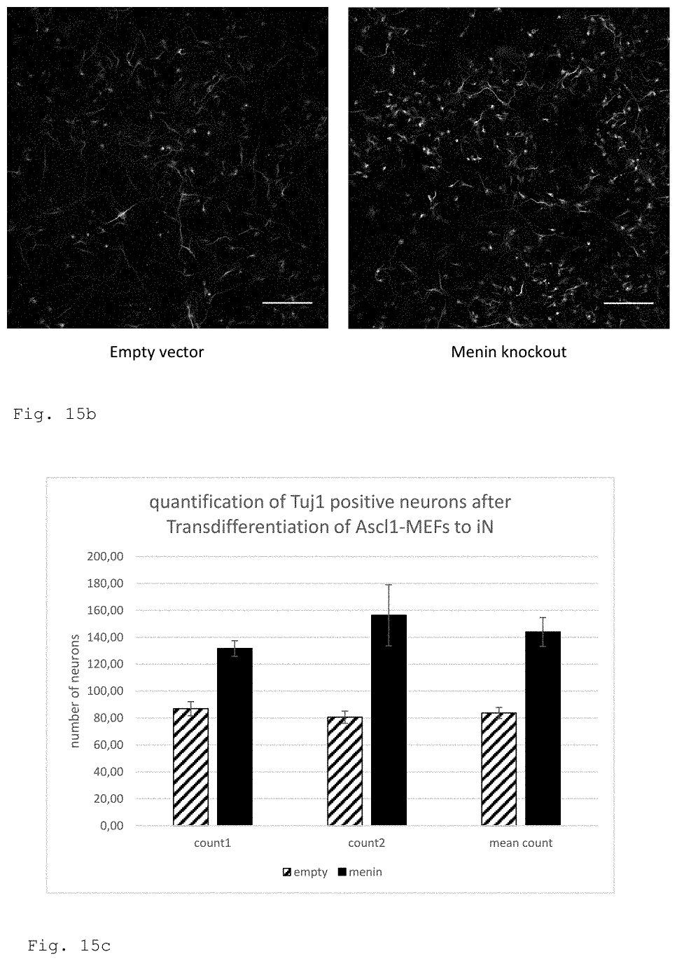

FIG. 15 shows cell images with and without menin knockout. The plot of FIG. 15 illustrates the difference in IN number obtained from Ascl1 cell line after menin knockout and empty guide control. Experiments were performed as biological triplica.

The data confirms the results of example 16 that neuronal transdifferentiation using neuronal transcription factors Ascl1 can be enhanced by Menin inhibition with different starting cells.

### Example 18: Results Summary

We applied CRISPR-UMI to a sensitizer screen for etoposide and identified all the expected genes in the NHEJ pathway, as well as unanticipated genes such as the transporter Abaci and the SUMO E3 ligase Zfp451 both by conventional and CRISPR-UMI analysis. Interestingly, mutations in Zfp451 nave recently been associated with cellular stress including DMA damage, however SL direct role in DNA damage resistance had not been reported. We therefore propose that chemical inhibition of Zfp451 will show strong synergy with etoposide in rapidly cycling tumor cells. CRISPR-UMI uncovered additional hits, Rad9a and Erbb4, that have previously been associated with DNA damage response and were not identified using the conventional analysis due to multiple single outlier clones that dominate sequencing space. Elimination of such outliers in conventional analysis also removed putatively false positive nits such as Trim71 and Eli, from the top scoring list. Furthermore, depletion levels based on median clone depletion—in particular for efficient guides—is more accurate to predict true biological effects compared to classical analysis suffering from a conceptual maximal level of measurable depletion.

Furthermore, CRISPR-UMI allowed us to score the number of independent IPS cell colonies formed in a single screen, and thus to identify well-known as well as new roadblocks of reprogramming. Importantly, the expected roadblocks of reprogramming Dotl1and Socs3 mostly scored with increased incidence, i.e. colony number, and would have been potentially missed in only read-based analysis. CRISPR-UMI identified Pias1, an E3 ligase of SUMOylation, and Menin, neither of which were previously implicated in IPS reprogramming. Interestingly, loss of Menin has been associated with facilitating of other lineage identity switches such as in vivo transdifferentiation of glucagon expressing cells to insulinomas potentially pointing to a more general role in maintenance of lineage identity.

By studying effect size and number, i.e. the number of independently iPS cell colonies and the read numbers obtained from each event, we can predict biological function directly from NGS data obtained from the screen. CRISPR-UMI identified conditions resulting primarily in faster reprogramming and thus bigger individual iPS colonies as confirmed by validation experiments. Examples are sgRNAs targeting Axin, APC and Tcf171, that lead to few but very big iPSC colonies. Indeed, Axin together with APC forms a destruction complex of beta-catenin negatively regulating Wnt signaling and acts through early promotion of endogenous pluripotency gene expression. This complex is often targeted via the Gsk3 inhibitor CHIR99021 to enhance reprogramming. Also, Tcf711 (sometimes referred to as Tcf3) inhibition was previously described to specifically promote early reprogramming stages by functioning as a transcriptional repressor of Wnt targets. In contrast to the Wnt signaling axis, targeting of Dot1l, Socs2, and Senp1 resulted primarily in an increased number of independent UMIs with few reads representative of small iPS cell colonies. The probability of a fibroblast cell to dedifferentiate into an iPS colony in the transgenic system, we used is typically below 1%. Therefore, rather than affecting kinetics, these genes modulate the likelihood of iPS colony formation. The ubiquitin E3 ligase Socs3 was previously reported as negative regulator of Stat3 signaling. Thus, Socs3 knockout boosts Stat3 signaling downstream of LIE, potentially explaining the increased numbers of UMIs we observed. However, Socs3 knockout also led to increased differentiation to trophoblast giant cells in our validation resulting in low absolute numbers of iPS cells, in line with previous observations that Socs3 results in differentiation of trophoblast stem cells to giant cells. Taken together we hypothesize that Socs3 knockout boosts LIF-induced Stat3 signaling which leads to increased reprogramming towards iPSCs but then also induces differentiation to trophoblast giant cells resulting in the observed small cell numbers/unique molecular identifier. Similarly, targeting of Dot1l also resulted mostly in increased number of independent colonies. These observations demonstrate the additional insights uncovered by positive selection screens. Moreover, because we can count the number and quantify the effect incidents, this method can detect, clonal effects in positive selection screens and thereby avoid false positive calls by clonal outgrowth, e.g. due to double infection of one cell with 2 guide vehicles whereby one positively selecting guide also enriches for another passenger guide.

Out studies were further validated by generating human iPS cells using menin suppression or editing, thereby ablating menin activity, iPS cells behave like ESCs and could be reprogrammed to neuronal cells.

1. A method of preparing a population of iPS cells comprising: (i) expressing one or more Yamanaka factors selected from Oct3/4, Sox2, Klf4, Myc, Nanog and Lin28; and (ii) reducing the amount and/or activity of Menin (Men1) in a population of target cells.

2. A method of enhanced differentiation of a first cell into a somatic cell of a tissue of interest, comprising: (i) treating a cell with a differentiation factor of said tissue of interest-; and (ii) reducing the amount and/or activity of Menin (Men1) in a population of target cells; wherein (A) said first cell is a cell of low transdifferentiation capacity selected from an adult or mature dermal cell, a blood cell, a hair follicle cell or a urinary cell; or (B) said differentiation is to a somatic cell of a different germ layer than the first cell; or (C) said somatic cell is a non-cardiac cell.

3. The method of claim 1, wherein the step of reducing the amount of Menin comprises administering to the cells one or more agents that inhibit the expression of Menin, preferably wherein the one or more agents are inhibitory nucleic acids.

4. The method of claim 3, wherein the inhibitory nucleic acid is a siRNA, shRNA, sgRNA, miRNA or antisense nucleic acid molecule.

5. The method of claim 3, wherein the agent is an inhibitory siRNA, shRNA, sgRNA, miRNA or antisense nucleic acid molecule encoded by a transient expression system in the target cells.

6. The method of claim 5, wherein the target cell is exposed to a transient expression system for between 36 to 120 hours.

7. The method claim 1, wherein the step of reducing the activity of Menin comprises administering to the cells one or more agents that inhibit the activity of Menin.

8. The method of claim 7, wherein the one or more activity inhibiting agents are antibodies, inhibitory ligands of Menin, inhibitory mimics of Menin, or small molecule inhibitors transiently inhibiting the activity of Menin, preferably wherein the small molecule inhibitors are selected from KO-382, MI-3, MI-2, MI-463, MI-503, Vinpocetine, MI-136 and/or Sinomenine.

9. The method of claim 1, wherein the step of expressing one or more Yamanaka factors or of treating a cell with a differentiation factor, respectively, comprises integrative approaches, preferably retroviral, lentiviral or adenoviral expression vectors, especially excisable and inducible vectors, or non-integrative approaches, preferably integration-defective viral, episomal, RNA or protein delivery techniques, preferably nonviral vector-based IVT-mRNA nanodelivery systems, in particular preferred, wherein the integrative or non-integrative approach for expressing one or more Yamanaka factors is transient or inducible.

10. The method of claim 1, wherein the method additionally comprises reducing the activity of Pias1 in the target cells.

11. The method claim 1, comprising isolating the iPS cells or the somatic cell, respectively, from the target cell population.

12. The method of claim 1, wherein the target cells are somatic mammalian cells; preferably, human cells, non-human primate cells, or mouse cells; and/or preferably wherein the somatic mammalian cells are fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells.

13. The method of claim 1, wherein reducing the amount and/or activity of Menin (Men1) enhances reprogramming of the target cell to an iPS cell by the expression of the one or more Yamanaka factors selected from Oct3/4, Sox2, Klf4, Myc, Nanog and Lin28.

14. The method of claim 1, wherein the method comprises (i) expressing one or more Yamanaka factors selected from Oct3/4, Sox2, Klf4, Myc, Nanog and Lin28, and (ii) reducing the amount and/or activity of Menin (Men1), together.

15. A method for preparing a population of differentiated cells, comprising: (i) preparing a population of iPS cells according to the method of claim 1; and (ii) differentiating the iPS cells using a protocol or factor to form a population of differentiated cells.

16. A population of iPS cells prepared according to the method of claim 1, wherein the amount and/or activity of Menin is reduced compared to iPS cells that have not been treated with a Menin-reducing agent.

17. The population of iPS cells according to claim 16, wherein the cells comprise an inhibitory nucleic acid molecule of Menin.

18. A kit for enhanced reprogramming of somatic cells into iPS cells comprising: one or more Yamanaka factors or one or more Yamanaka-inducing agents and one or more agents that inhibit the expression, translation or activity of Menin, preferably wherein said Yamanaka factors or Yamanaka-inducing agents and one or more agents that inhibit the expression, translation or activity of Menin are in one or more cell culture medium.

19. The kit of according to claim 18, further comprising an inhibitory nucleic acid molecule of Menin, preferably with a transient transfection agent, preferably a non-integrating virus or an episomal vector.
